(19) United States
(12) Patent Application Publication
(10) Pub. No.: US 2013/0210901 A1
(43) Pub. Date: Aug. 15, 2013

(54) METHOD OF TREATING NEURODEGENERATIVE DISEASES

(71) Applicants: Hermona Soreq, Jerusalem (IL); Amit Berson, Jerusalem (IL)

(72) Inventors: Hermona Soreq, Jerusalem (IL); Amit Berson, Jerusalem (IL)

(73) Assignee: Yissum Research Development Company of the Hebrew University of Jerusalem Ltd., Jerusalem (IL)

(21) Appl. No.: 13/825,131

(22) PCT Filed: Sep. 20, 2011
(86) PCT No.: PCT/IL2011/000743

§ 371 Date: Mar. 20, 2013

Related U.S. Application Data
(60) Provisional application No. 61/384,424, filed on Sep. 20, 2010.
(60) Provisional application No. 61/497,670, filed on Jun. 16, 2011.

(51) Int. Cl. C07H 21/02

(52) U.S. Cl. CPC C07H 21/02
USPC 514/44 R

(57) ABSTRACT

An agent which upregulates an amount or activity of a heterogeneous nuclear ribonucleoproteins A/B (hnRNP A/B) polypeptide is disclosed, for use in treating a neurodegenerative disease. Pharmaceutical compositions comprising same are also disclosed.

### FIELD AND BACKGROUND OF THE INVENTION

The present invention, in some embodiments thereof, relates to methods of treating sporadic neurodegenerative diseases and, more particularly, but not exclusively, to methods of treating Alzheimer's disease.

Alzheimers Disease (AD) is the leading cause of dementia, accounting for 50-80% of dementia cases and afflicting 35 million world-wide. The vast majority of patients are sporadic cases, but none of the currently recognized causes of AD can fully explain to the disease phenotype. For example, 40% of amyloid plaque pathology carriers do not develop AD and amyloid-independent mechanisms have been recently acknowledged.

Roughly 50% of known debilitating mutations in RNA-binding proteins cause neuronal-related diseases. For example, mutations in the RNA/DNA binding factors TDP-43 and TLS/FUS which are structurally related to the family of heterogeneous nuclear ribonucleoproteins (hnRNPs), induce familial amyotrophic lateral sclerosis. Also, mutations in SMN2 induce spliceosome mis-assembly and motor neuron degeneration in spinal muscular atrophy, further corroborating the link between RNA splicing and neuronal survival.

Specifically, members of the hnRNPA/B family, namely hnRNP A1 and A2/B2 are among the most abundant nuclear proteins. The modular structure of hnRNPs A/B contains several RNA binding motifs spanning RNA recognition motifs (RRM), KH domains and arginine-glucine-glycine (RGG) boxes. SMN2 exon 7 splicing was shown to specifically repressed by hnRNP A1; recently, hnRNPA1 expression was shown to increase in peripheral blood mononuclear cells from AD patients, and hnRNP A1 polymorphisms have been linked to frontotemporal lobar degeneration (FTLD). Also, auto-antibodies against hnRNP A1 have been identified in human T-lymphotropic virus type 1 (HTLV-1)-associated myelopathy/tropical spastic paraparesis (HAM/TSP), a disease that can be indistinguishable from multiple sclerosis (MS). That such auto-antibodies stained neurons and inhibited neuronal firing in brain slices, suggests that hnRNP A1 is critical for neuronal function.

In several engineered mouse models of human diseases, transcriptome profiling demonstrated alternative splicing perturbations (Du et al.; Zhang et al., 2008), suggesting that global changes in the repertoire of mRNA variants may be involved in the initiation and/or progression of neurodegenerative diseases. However, neither the human nor the mouse studies could indicate if splicing impairments may be involved in sporadic neurodegeneration as well.

### SUMMARY OF THE INVENTION

According to an aspect of some embodiments of the present invention there is provided an agent which upregulates an amount or activity of a heterogeneous nuclear ribonucleoproteins A/B (hnRNP A/B) polypeptide, for use in treating a neurodegenerative disease.

According to an aspect of some embodiments of the present invention there is provided a method of treating a neurodegenerative disease in a subject in need thereof comprising administering to the subject a therapeutically effective amount of an agent which upregulates an amount or activity of heterogeneous nuclear ribonucleoproteins A/B (hnRNP A/B) in a brain of the subject, thereby treating the neurodegenerative disease.

According to an aspect of some embodiments of the present invention there is provided a pharmaceutical composition comprising miR132 and/or a miR211 antagonist as an active agent and a pharmaceutically acceptable carrier. According to some embodiments of the invention, the hnRNP A/B polypeptide is hnRNP A1 and/or A2/B 1.

According to some embodiments of the invention, the agent comprises a polynucleotide agent.

According to some embodiments of the invention, the polynucleotide agent comprises a miRNA.

According to some embodiments of the invention, the miRNA is encoded by a sequence as set forth in SEQ ID NO: 86.

According to some embodiments of the invention, the polynucleotide agent comprises a miRNA antagonist.

According to some embodiments of the invention, the miRNA antagonist comprises a miR211 antagonist.

According to some embodiments of the invention, the miR211 antagonist comprises a sequence as set forth in SEQ ID NO: 87.

According to some embodiments of the invention, the neurodegenerative disease is selected from the group consisting of Alzheimer's disease, epilepsy, amyatrophic lateral sclerosis, stroke, autoimmune encephalomyelitis, diabetic neuropathy and glaucomatous neuropathy.

According to some embodiments of the invention, the neurodegenerative disease is Alzheimer's disease.

According to some embodiments of the invention, the pharmaceutical composition is formulated for crossing the blood brain barrier.

According to some embodiments of the invention, the miR211 antagonist comprises a sequence as set forth in SEQID NO: 87.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings and images. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-G illustrate that global alternative splicing alterations associate with drastic reductions in hnRNP A/B in the entorhinal cortex of AD patients. (A) Venn diagram of gene expression and alternative splicing changes between Alzheimer (AD) patients and non-demented controls (CT). (B) Majority of splicing events in AD involved exon inclusions. Kolmogorov-Smirnov test: p<0.0001. (C) RT-PCR and (D) real-time RT-PCR alternative splicing analysis. *p<0.05, Student's t-test, n=7 for both groups. (E) hnRNP A/B Immunohistochemistry of human entorhinal cortices. Scale bar=50 nm, in inset=25 nm. *p<0.05, Student's t-test. CT n=5, AD n=10. (F) Immuno-blotting of entorhinal cortex homogenates shows drastic reductions of hnRNP A1, A2/B1 and A3 in AD. Columns: densitometric analysis. *p<0.05, Student's t-test, n=6 for both groups. (G) Immuno-blotting of entorhinal cortex homogenates shows no changes in SR protein levels in AD. Columns: densitometric analysis. p=n.s, Student's t-test, n=6 for both groups.

FIGS. 2A-E relate to global alternative splicing alterations in AD patients. (A) Gene ontology analysis of alternative splicing and gene expression changes discover known and new pathways involved in neurodegeneration. Top, Alternative splicing analysis. Bottom, gene expression analysis. All categories shown are enriched over background. p-value<0.05. (B) No changes were observed in the levels of SR protein members or SR protein kinases. n=6, p>0.05 (C) No change in the mRNA levels of hnRNP A1, A2/B1 and A3 and the RNA levels of the major and minor spliceosomal components U1, U2 and U11, U12. Student's t-test: p=NS. n=6 in each group (D) triple labeling of hnRNP A/B (red), astrocytes (GFAP, green) and nuclei (DAPI, blue) demonstrates low basal levels of hnRNP A/B in astrocytes and validates reduction of neuronal hnRNP A/B. (E) non-significant increase of hnRNP A/B in Parkinson's disease substantia nigra pars compacta. Mann-Whitney U-test p=NS, CT n=4, PD n=5.

FIGS. 3A-G illustrate that knockdown of hnRNP A/B impairs learning and memory and alters cortical network connectivity. (A) Selectivity of lentiviral shRNA-mediated knockdown of hnRNP A1 and/or A2/B1 mRNA in primary cortical cultures. *p<0.05 Student's t-test, n=6, performed in duplicates (B) hnRNP A/B knock-down mice show higher escape latencies in the Morris water maze. Two-way ANOVA revealed effects of trial number (F_{11,426}=2.26, p<0.02) and treatment (F_{3,426}=6.65, p<0.001). LSD post-hoc comparison: vs. shA1 p<0.01, shA2 p<0.01, A1+2 p<0.01, n=10 in each group. (C) hnRNP A/B knock-down mice spent less time in the correct quadrant in the probe test. One-way ANOVA: F_{3,36}=9.90, p<0.001; LSD post-hoc comparison: vs. shA1 *p<0.01, shA2 “p<0.001, A1+2 “p<0.001. (D) Examples of swimming patterns (correct quadrant highlighted). (E) ECoG recordings were continuously performed for 4 days from the entorhinal cortex of behaving shA1+2 and shCT mice. Fourier transform was performed to derive the power spectrum of each minute, after which all minuets were averaged for each animal in the frequency space. Power spectrums for 3 mice of each group were averaged and statistically analyzed. Representative examples are shown. Bar graph insets are summation of the area below each line for each range. n=3, *p<0.05, Student's t-test. (F) Immuno-blots of hnRNP A/B and synaptophysin in primary cortical neurons following single or double hnRNP A/B knock-down. Note specific reductions in hnRNP A1 or A2/B1. (G) Reduced activity of secreted acetylcholinesterase in knock-down cultures. N=4 P<0.05.

FIGS. 4A-B are graphs related to the knockdown of hnRNP A/B. (A) Lenti-viral injected mice were tested in the Rotarod test of motor coordination: all four groups of mice performed similarly. Two-way ANOVA revealed a significant effect of trial number (F_{2,108}=3.66, p<0.03), but no significant effect of treatment (F_{3,108}=1.8, p=NS) and no significant interaction (F_{6,108}=0.03, p=NS). Average time in seconds ±SEM is shown. (B) “Amount of motor activity” was defined as the area below the normalized squared signal of the activity channel for the daily recording duration and that of the night. There was no change between EC-shRNA A1+A2/B1 and control mice in amount of activity during the night nor during the day. If the activity channel indeed reports motorically active epochs, one would expect to see more activity during the night in these nocturnal animals. A significant change in the expected direction was seen for each group separately, and for the two groups combined together, as seen in this figure (t-test p-value=0.009). Another strengthening for the validity of the activity channel is the clear sleep/wakefulness patterns in the power spectrums that were observed (as seen in FIG. 3E) when defining sleep/wakefulness epochs using this measure.

FIGS. 5A-I illustrates that hnRNP A/B knockdowns induce neuronal dendrite and synapse loss, with aberrant alternative splicing. (A) No changes in activated caspase 9 (red). Scale bar=50 μm (B) Reduced dendrites density (green, MAP2) and synapse loss (pre-synapses,red, synaptophysin) after hnRNP A/B knockdown in primary cortical neurons. Scale bar=50 μm (C) Reduced number of synapses marked with synaptophysin in A/B shRNA treated neurons. One-way ANOVA: F_{3,31}=4.31, p<0.05; LSD post-hoc: vs. shA1 *p<0.05, shA2 **p<0.01, A1+2 *p<0.05. (D) Reduced dendritic density in shRNA A/B treated neurons. One-way ANOVA: F_{3,32}=16.21, p<0.001; LSD post-hoc: vs. shA1 *p<0.001, shA2 *p<0.001, A1+2 *p<0.001. (E) Magnified single dendrites and synapses from (B) Scale bar=10 μm. (F) Synaptophysin puncta are smaller in shRNA A/B treated neurons. p<0.05 Kolmogorov-Smirnov test. (G) Reduced activity of secreted acetylcholinesterase (AChE) in knockdown cultures. Kruskal-Wallis test: H_{3,16}=8.62, p<0.05. Mann-Whitney U-test: vs. shA1 *p<0.05, shA2 *p<0.05, A1+2 *p<0.05. (H) Immuno-blots of hnRNP A/B, synaptophysin and neuroligin 3 in primary cortical neurons following hnRNP A/B knockdown. (I) Alternative splicing events in primary cultures following hnRNP A/B knockdown. Wilks' Lambda test: Rao's R_{3,33}=20, p<0.05. LSD Post hoc *p<0.05.

FIGS. 6A-B illustrate that medium of treated cultures demonstrates no induction of cell death following knock-down of hnRNP A1, A2/B1 or both as quantified by activated caspase 9 (FIG. 6A) and lactate dehydrogenase activity (FIG. 6B). (A) Student's t-test: p=NS, n=18 (B) Mann-Whitney U-test: p>0.05, n=3, performed in triplicates.

FIGS. 7A-L illustrate that Mouse hnRNPs A/B levels are modulated by cholinergic signaling but not by Aβ, Tau or aging. (A) hnRNPs A/B levels are similar in APPsw/PS1ΔE9 and non-transgenic siblings in pre- and post-symptomatic mice. n=3 for each group, Mann-Whitney U-test: p=NS. Top panel shows expression of human APP. (B) Immunofluorescence demonstrates no changes in hnRNPs A/B levels in cortices of double mutated K257T/P301S Tau mice. n=6, p=NS, Student's t-test. Scale bar=50 μm. (C) The cholinergic toxin mu p75-sap reduces hnRNPs A/B levels in the entorhinal cortex one month following injections. n>4, Mann-Whitney U-test, *p<0.05 (D) Real-time RT-PCR evidence in mu P75-sap injected mice of recapitulated alternative splicing events observed in the entorhinal cortex of AD patients. N>4 Mann-Whitney U-test, *p<0.05, performed in duplicates. (E) hnRNPs A/B are increased in primary neurons following addition of 10 μM of the cholinergic agonist carbachol to the culture medium for 48 hours. Student's t-test: n=6, *p<0.05 (F) Working hypothesis: miRNAs may be the missing link mediating reduced cholinergic signaling and altered hnRNP A/B expression in AD. (G) The 3′ UTRs of hnRNP A1, A2/B1 and A3 encompass a common seed region complementary to miR-211 (hnRNPA1 sequence, SEQ ID NO: 88—UGAAGUUCACCAUAAAAGGGAU; A2/B1 sequence, SEQ ID NO: 89—GCACUCUUUAAAAUAAAAGGGAA; hnRNPA3 sequence, SEQ ID NO: 90—CCAUUUAAAUUCUGAAAAGGGAU; miRNA 211 sequence, SEQ ID NO: 91—UCCGCUUCCUACUGUUUCCCUU. (H) Selective miRNA alterations in AD. miR-211, miR-132 and miR-204 levels in the entorhinal cortex of AD patients and controls. Expression normalized to sno-135. *p<0.05, Student's t-test, n=7 for both groups. Horizontal lines represent averages. (I) Antisense blockage of miR-211 using a locked nucleic acid complementary oligonucleotide increases hnRNP A/B expression in cultured N9 cells, *p=0.05, Mann-Whitney U-test, n=3. (J) 48 hours post pilocarpine injection the temporal cortex was dissected and miRNA expression was analyzed using RT-PCR. % expression from control±SEM, *p<0.05, Student's t-test. n=7 in each group. (K) miRNA-132 regulates miRNA-211 expression in primary neurons. Lentiviral delivery of miRNA-211 (Lenti211) and antisense oligonucleotides complementary to miRNA-132 (AM132) were used to alter miRNA expression in primary neurons. *p<0.05, Mann-Whitney U-test, n≧4. Note that miRNA-211 overexpression did not affect miRNA-132 levels. (L) Immunofluorescence demonstrates no changes in hnRNPs A/B levels in entorhinal cortices of APPsw/PS 1ΔE9 and double mutated K257T/P301S Tau mice. N>3, P>0.05.

FIGS. 8A-B illustrate that there is a negative correlation between miR-211 and hnRNP A/B during development. hnRNP A/B are reduced (8A) while miR-211 is upregulated (8B) during post-natal cortex development in mice. Post natal days 1 (P1) to 19 (P19) are shown. Kruskal-Wallis test: H_{3,12}=7.83, p<0.05. Mann-Whitney U-test: P1 vs. P6 *p<0.05, p12 *p<0.05, p19 *p<0.05.

FIGS. 9A-B illustrate biological titer determination. 9A: 293HEK cells were infected with serially diluted GFP expressing lentivirus and infecting units/ml were calculated as described in the figure. 9B: examples of primary neurons infected with GFP-lentivirus

### DESCRIPTION OF SPECIFIC EMBODIMENTS OF THE INVENTION

The present invention, in some embodiments thereof, relates to methods of treating sporadic neurodegenerative diseases and, more particularly, but not exclusively, to methods of treating Alzheimer's disease.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

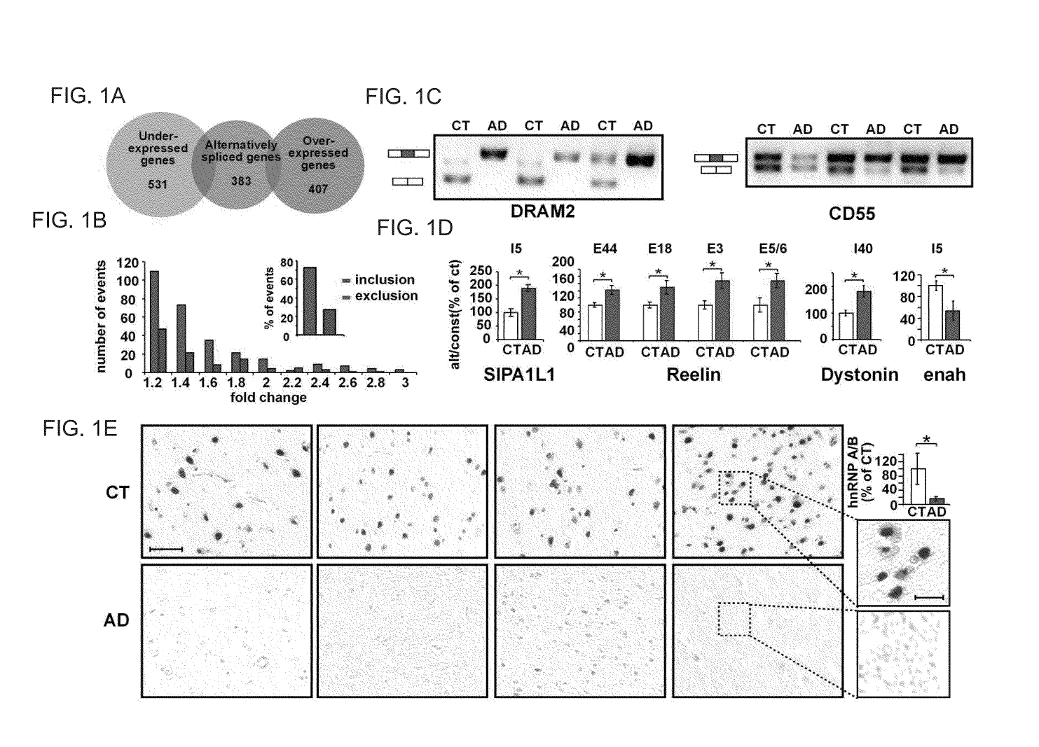

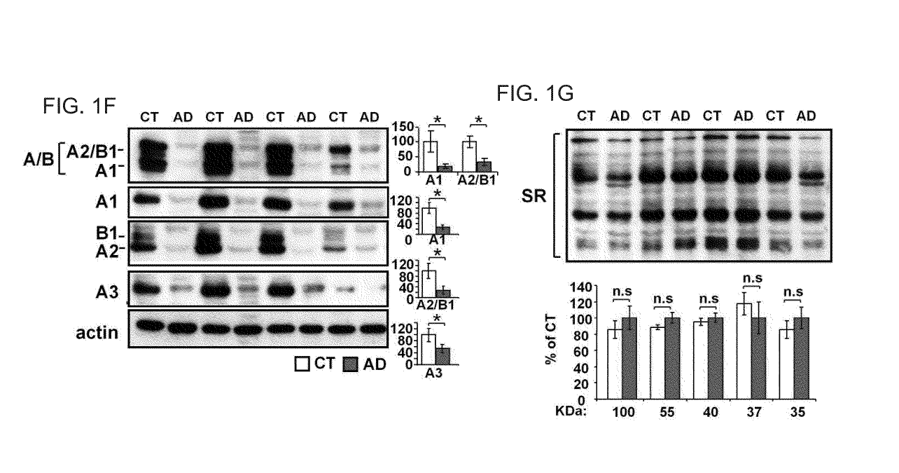

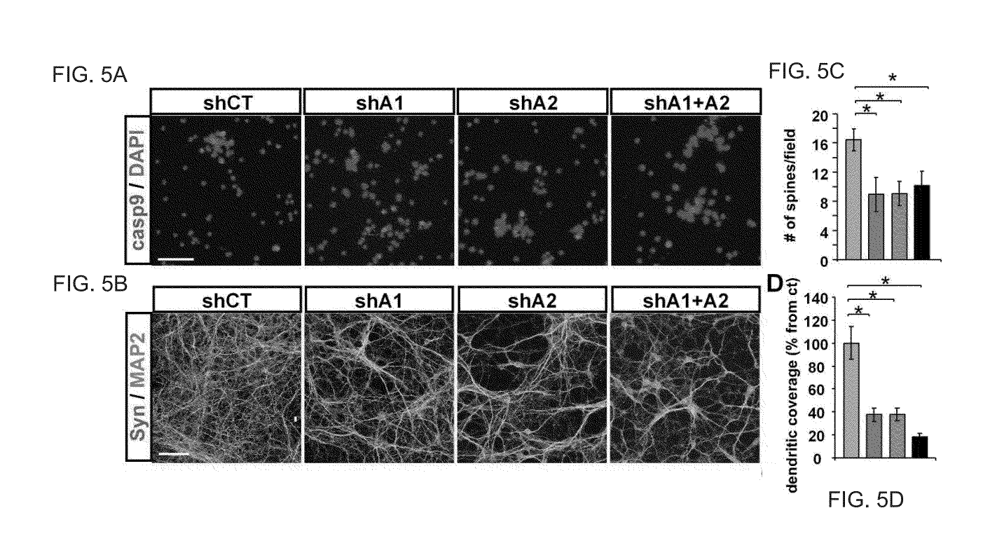

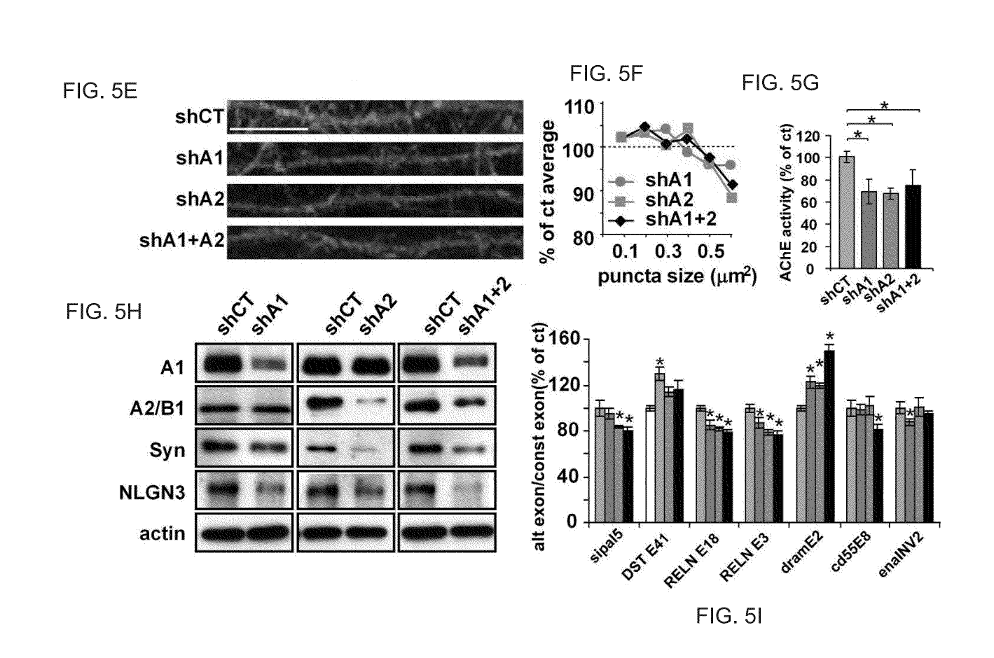

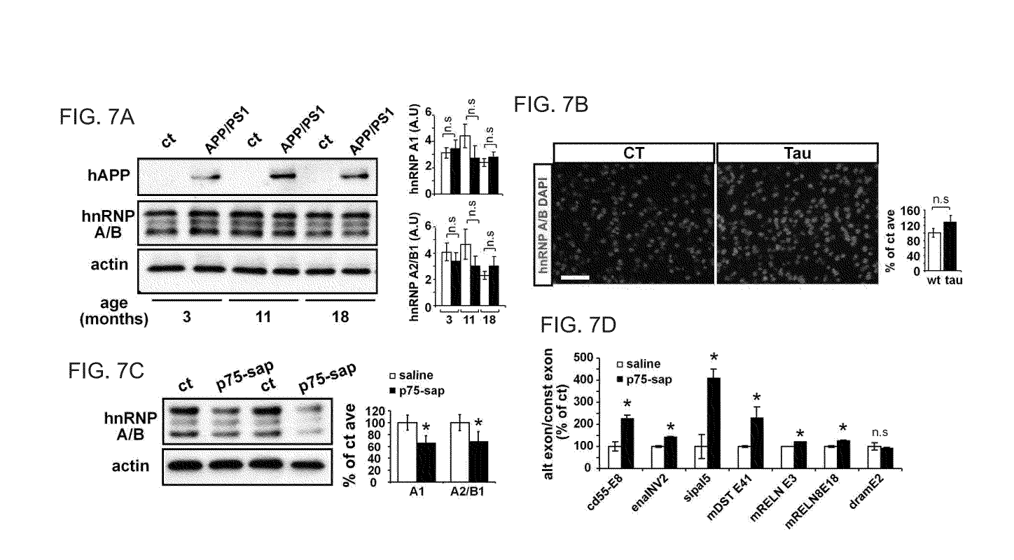

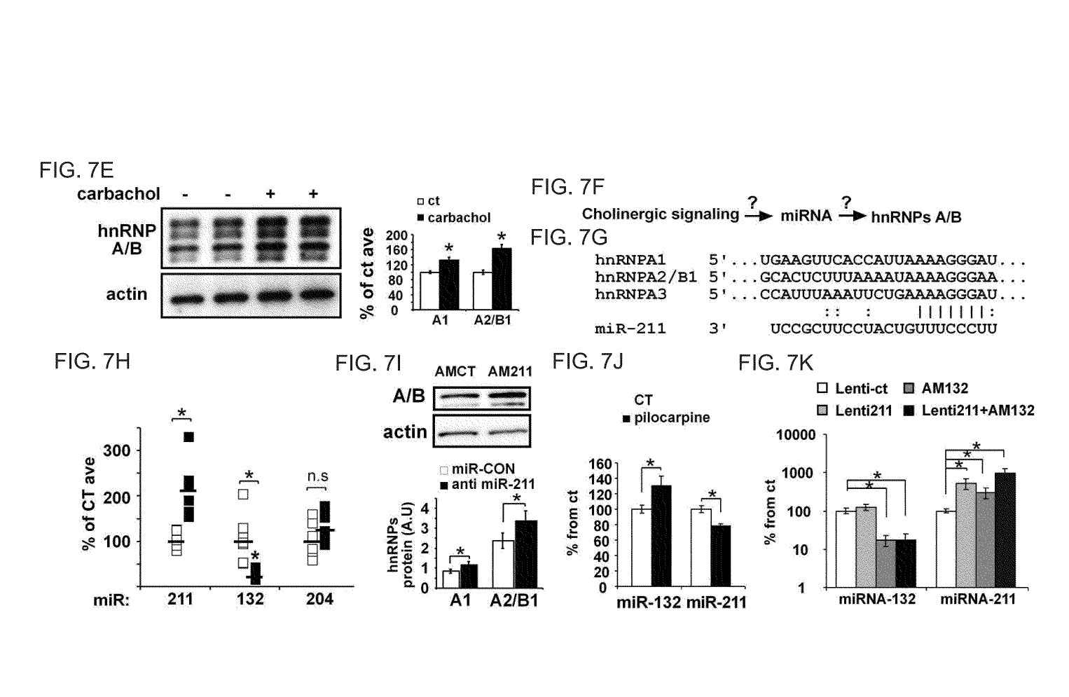

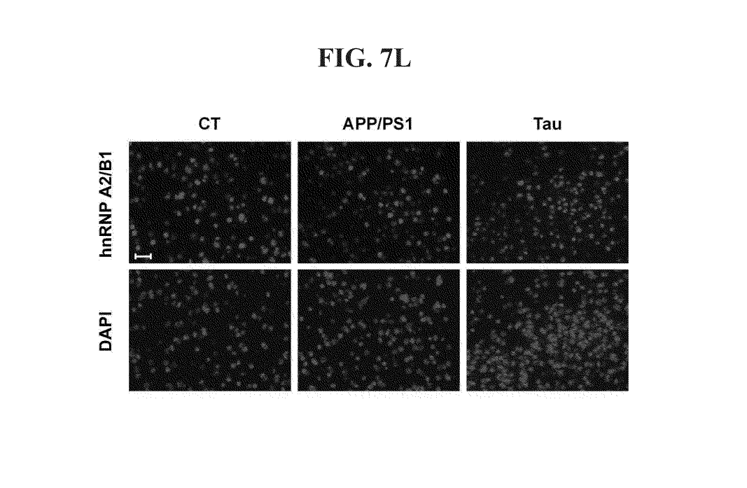

Alzheimer' s disease (AD) notably involves failed synaptic functioning and to premature death of cholinergic neurons, but the underlying mechanism(s) and possible interrelationships between these two phenomena are yet incompletely understood. Using a global high-throughput screening, the present inventors discovered increased exon inclusion events and corresponding decreases in the exon exclusion regulators, heteronuclear ribonucleoprotein particles (hnRNPs) in the entorhinal cortex from AD patients compared to non-demented controls (FIGS. 1B, 1F and 1G). This was accompanied by increased microRNA (miR-211) which co-targets three different hnRNP mRNAs (FIGS. 7G-H); and lentiviral-mediated knockdown of these hnRNPs caused synapse loss in cultured neurons and learning and memory impairments in brain-injected mice (FIGS. 5A-I). Furthermore, in vivo destruction of cholinergic neurons, but not APP or TAU mutations, reduced brain hnRNP levels, and the synaptogenesis regulating and acetylcholinesterase (AChE)-targeted miR-132 was drastically reduced in the AD entorhinal cortex, possibly attributing part of the loss of cholinergic input to this change. Together, these findings suggest that AD involves a feed-forward loop of miR-211 increases which mediate hnRNP depletion, leading to synapse loss; and parallel miR-132 decreases which cause AChE elevation, impair cholinergic signaling and enhance neuroinflammation while exacerbating hnRNPs loss.

Thus, according to one aspect of the present invention, there is provided a method of treating a disease in a subject in need thereof comprising administering to the subject a therapeutically effective amount of an agent which upregulates an amount or activity of heterogeneous nuclear ribonucleoproteins A/B (hnRNP A/B) in a brain of the subject, thereby treating the disease.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition/disease, substantially ameliorating clinical or aesthetical symptoms of a condition/disease or substantially preventing the appearance of clinical or aesthetical symptoms of a condition/disease.

According to one embodiment, the agents of this aspect of the present invention are used to treat neurodegenerative diseases.

Exemplary neurodegenerative diseases that may be treated according to this aspect of the present invention include neurodegenerative diseases including, but not limited to Alzheimer's disease, epilepsy, amyatrophic lateral sclerosis, stroke, autoimmune encephalomyelitis, diabetic neuropathy and glaucomatous neuropathy.

According to a particular embodiment, the neurodegenerative disorder is not Parkinson's disease.

The present invention further contemplates use of the agents described herein for treating the cognitive decline after cardiac surgery and also for the treatment of inflammatory conditions, as described herein below.

Inflammatory diseases—Include, but are not limited to, chronic inflammatory diseases and acute inflammatory diseases.

Inflammatory diseases associated with hypersensitivity Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann NY Acad Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost.2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like beta-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 to (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann NY Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

Autoimmune Diseases

Include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost.2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. diseases include, but are not limited to autoimmune diseases of the pancreas, Type 1 diabetes (Castano L. and Eisenbarth G S. Ann Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander RB. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau YE. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326). Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Bane syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann NY Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to to, ear diseases, autoimmune ear diseases (Yoo T J. et al., Cell Immunol 1994 August; 157 (1):249) and autoimmune diseases of the inner ear (Gloddek B. et al., Ann NY Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

### Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Graft Rejection Diseases

Examples of diseases associated with transplantation of a graft include, but are not limited to, graft rejection, chronic graft rejection, subacute graft rejection, hyperacute graft rejection, acute graft rejection and graft versus host disease.

Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Cancerous Diseases

Examples of cancer include but are not limited to carcinoma, lymphoma, blastoma, sarcoma, and leukemia. Particular examples of cancerous diseases but are not limited to: Myeloid leukemia such as Chronic myelogenous leukemia. Acute myelogenous leukemia with maturation. Acute promyelocytic leukemia, Acute nonlymphocytic leukemia with increased basophils, Acute monocytic leukemia. Acute myelomonocytic leukemia with eosinophilia; Malignant lymphoma, such as Birkitt's Non-Hodgkin's; Lymphoctyic leukemia, such as Acute lumphoblastic leukemia. Chronic lymphocytic leukemia; Myeloproliferative diseases, such as Solid tumors Benign to Meningioma, Mixed tumors of salivary gland, Colonic adenomas; Adenocarcinomas, such as Small cell lung cancer, Kidney, Uterus, Prostate, Bladder, Ovary, Colon, Sarcomas, Liposarcoma, myxoid, Synovial sarcoma, Rhabdomyosarcoma (alveolar), Extraskeletel myxoid chonodrosarcoma, Ewing's tumor; other include Testicular and ovarian dysgerminoma, Retinoblastoma, Wilms' tumor, Neuroblastoma, Malignant melanoma, Mesothelioma, breast, skin, prostate, and ovarian.

As mentioned, the agents of the present invention upregulate an amount or activity of heterogeneous nuclear ribonucleoproteins A/B (hnRNP A/B).

As used herein, the term “heterogeneous nuclear ribonucleoprotein A/B (hnRNP A/B)” refers to the expression product (either RNA or protein) of the human gene located at chromosomal location 7p15 (gene ID 3181).

The hnRNPs are RNA binding proteins and they complex with heterogeneous nuclear RNA (hnRNA). These proteins are associated with pre-mRNAs in the nucleus and appear to influence pre-mRNA processing and other aspects of mRNA metabolism and transport. The protein encoded by this gene has two repeats of quasi-RRM domains that bind to RNAs. This gene has been described to generate two alternatively spliced transcript variants which encode different isoforms [NM_{—}002137—SEQ ID NO: 92 and NM_{—}031243.2—SEQ ID NO: 93]. Variant (B1) contains an additional 36 bases compared to variant A2. This additional region affects only the beginning of the coding region. The N-terminus of isoform B1 is thus different from isoform A2.

The agents of the present invention can be any molecule effective for its intended use, including, but not limited to, chemicals, antibiotic compounds known to modify gene expression, modified or unmodified polynucleotides (including oligonucleotides), polypeptides, peptides, small RNA molecules, micro-RNAs and anti-micro-RNAs. Preferably, the agent used by the present invention is a polynucleotide agent.

According to a particular embodiment, the agent is one (or is formulated in such a way, as further described herein below) which is capable of crossing the blood brain barrier.

The term “polynucleotide” refers to a single-stranded or double-stranded oligomer or polymer of ribonucleic acid (RNA), deoxyribonucleic acid (DNA) or mimetics thereof. This term includes polynucleotides and/or oligonucleotides derived from naturally occurring nucleic acids molecules (e.g., RNA or DNA), synthetic polynucleotide and/or oligonucleotide molecules composed of naturally occurring bases, sugars, and covalent internucleoside linkages (e.g., backbone), as well as synthetic polynucleotides and/or oligonucleotides having non-naturally occurring portions, which function similarly to respective naturally occurring portions. The length of the polynucleotide of the present invention is optionally of 100 nucleotides or less, optionally of 90 nucleotides or less, optionally 80 nucleotides or less, optionally 70 nucleotides or less, optionally 60 nucleotides or less, optionally 50 nucleotides or less, optionally 40 nucleotides or less, optionally 30 nucleotides or less, e.g., 29 nucleotides, 28 nucleotides, 27 nucleotides, 26 nucleotides, 25 nucleotides, 24 nucleotides, 23 nucleotides, 22 nucleotides, 21 nucleotides, 20 nucleotides, 19 nucleotides, 18 nucleotides, 17 nucleotides, 16 nucleotides, 15 nucleotides, optionally between 12 and 24 nucleotides, optionally between 5-15, optionally, between 5-25, most preferably, about 20-25 nucleotides.

The polynucleotides (including oligonucleotides) designed according to the teachings of the present invention can be generated according to any oligonucleotide synthesis method known in the art, including both enzymatic syntheses or solid-phase syntheses. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Md.; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

It will be appreciated that a polynucleotide comprising an RNA molecule can be also generated using an expression vector as is further described hereinbelow.

Preferably, the polynucleotide of the present invention is a modified polynucleotide. Polynucleotides can be modified using various methods known in the art.

For example, the oligonucleotides or polynucleotides of the present invention may comprise heterocylic nucleosides consisting of purines and the pyrimidines bases, bonded in a 3′-to-5′ phosphodiester linkage.

Preferably used oligonucleotides or polynucleotides are those modified either in backbone, internucleoside linkages, or bases, as is broadly described hereinunder.

Specific examples of preferred oligonucleotides or polynucleotides useful according to this aspect of the present invention include oligonucleotides or polynucleotides containing modified backbones or non-natural internucleoside linkages. Oligonucleotides or polynucleotides having modified backbones include those that retain a phosphorus atom in the backbone, as disclosed in U.S. Pat. Nos.: 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Preferred modified oligonucleotide or polynucleotide backbones include, for example: phosphorothioates; chiral phosphorothioates; phosphorodithioates; phosphotriesters; aminoalkyl phosphotriesters; methyl and other alkyl phosphonates, including 3′-alkylene phosphonates and chiral phosphonates; phosphinates; phosphoramidates, including 3′-amino phosphoramidate and aminoalkylphosphoramidates; thionophosphoramidates; thionoalkylphosphonates; thionoalkylphosphotriesters; and boranophosphates having normal 3′-5′ linkages, 2′-5′ linked analogues of these, and those having inverted polarity wherein the adjacent pairs of nucleoside units are linked 3′-5′ to 5′-3′ or 2′-5′ to 5′-2′. Various salts, mixed salts, and free acid forms of the above modifications can also be used.

Alternatively, modified oligonucleotide or polynucleotide backbones that do not include a phosphorus atom therein have backbones that are formed by short-chain alkyl or cycloalkyl internucleoside linkages, mixed heteroatom and alkyl or cycloalkyl internucleoside linkages, or one or more short-chain heteroatomic or heterocyclic internucleoside linkages. These include those having morpholino linkages (formed in part from the sugar portion of a nucleoside); siloxane backbones; sulfide, sulfoxide, and to sulfone backbones; formacetyl and thioformacetyl backbones; methylene formacetyl and thioformacetyl backbones; alkene-containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

Other oligonucleotides or polynucleotides which may be used according to the present invention are those modified in both sugar and the internucleoside linkage, i.e., the backbone of the nucleotide units is replaced with novel groups. The base units are maintained for complementation with the appropriate polynucleotide target. An example of such an oligonucleotide mimetic includes a peptide nucleic acid (PNA). A PNA oligonucleotide refers to an oligonucleotide where the sugar-backbone is replaced with an amide-containing backbone, in particular an aminoethylglycine backbone. The bases are retained and are bound directly or indirectly to aza-nitrogen atoms of the amide portion of the backbone. United States patents that teach the preparation of PNA compounds include, but are not limited to, U.S. Pat. Nos. 5,539,082; 5,714,331; and 5,719,262; each of which is herein incorporated by reference. Other backbone modifications which may be used in the present invention are disclosed in U.S. Pat. No. 6,303,374.

Additionally, or alternatively the oligonucleotides/polynucleotide agents f the present invention may be phosphorothioated, 2-o-methyl protected and/or LNA modified.

Oligonucleotides or polynucleotides of the present invention may also include base modifications or substitutions. As used herein, “unmodified” or “natural” bases include the purine bases adenine (A) and guanine (G) and the pyrimidine bases thymine (T), cytosine (C), and uracil (U). “Modified” bases include but are not limited to other synthetic and natural bases, such as: 5-methylcytosine (5-me-C); 5-hydroxymethyl cytosine; xanthine; hypoxanthine; 2-aminoadenine; 6-methyl and other alkyl derivatives of adenine and guanine; 2-propyl and other alkyl derivatives of adenine and guanine; 2-thiouracil, 2-thiothymine, and 2-thiocytosine; 5-halouracil and cytosine; 5-propynyl uracil and cytosine; 6-azo uracil, cytosine, and thymine; 5-uracil (pseudouracil); 4-thiouracil; 8-halo, 8-amino, 8-thiol, 8-thioalkyl, 8-hydroxyl, and other 8-substituted adenines and guanines; 5-halo, particularly 5-bromo, 5-trifluoromethyl, and other 5-substituted uracils and cytosines; 7-methylguanine and 7-methyladenine; 8-azaguanine and 8-azaadenine; 7-deazaguanine and 7-deazaadenine; and 3-deazaguanine and 3-deazaadenine. Additional modified bases include those disclosed in: U.S. Pat. No. 3,687,808; Kroschwitz, J. I., ed. (1990),“The Concise Encyclopedia Of Polymer Science And Engineering,” pages 858-859, John Wiley & Sons; Englisch et al. (1991), “Angewandte Chemie,” International Edition, 30, 613; and Sanghvi, Y. S., “Antisense Research and Applications,” Chapter 15, pages 289-302, S. T. Crooke and B. Lebleu, eds., CRC Press, 1993. Such modified bases are particularly useful for increasing the binding affinity of the oligomeric compounds of the invention. These include 5-substituted pyrimidines, 6-azapyrimidines, and N-2, N-6, and O-6-substituted purines, including 2-aminopropyladenine, 5-propynyluracil, and 5-propynylcytosine. 5-methylcytosine substitutions have been shown to increase nucleic acid duplex stability by 0.6-1.2° C. (Sanghvi, Y. S. et al. (1993), “Antisense Research and Applications,” pages 276-278, CRC Press, Boca Raton), and are presently preferred base substitutions, even more particularly when combined with 2′-O-methoxyethyl sugar modifications. According to preferred embodiments of the present invention the modified polynucleotide of the present invention is partially 2′-oxymethylated, or more preferably, is fully 2′-oxymethylated.

According to one embodiment, upregulating the amount and/or activity of hnRNP A/B is effected by administering polynucleotides encoding the hnRNP A/B polypeptides and/or the polypeptides themselves.

DNA sequences are typically inserted into expression vectors to enable expression of the recombinant polypeptide or mRNA. The expression vector of the present invention includes additional sequences which render this vector suitable for replication and integration in prokaryotes, eukaryotes, or preferably both (e.g., shuttle vectors). Typical cloning vectors contain transcription and translation initiation sequences (e.g., promoters, enhances) and transcription and translation terminators (e.g., polyadenylation signals).

In addition to the elements already described, the expression vector of the present invention may typically contain other specialized elements intended to increase the level of expression of cloned nucleic acids or to facilitate the identification of cells that carry the recombinant DNA. For example, a number of animal viruses contain DNA sequences that promote the extra chromosomal replication of the viral genome in permissive cell types. Plasmids bearing these viral replicons are replicated episomally as long as the appropriate factors are provided by genes either carried on the plasmid or with the genome of the host cell.

The vector may or may not include a eukaryotic replicon. If a eukaryotic replicon is present, then the vector is amplifiable in eukaryotic cells using the appropriate selectable marker. If the vector does not comprise a eukaryotic replicon, no episomal amplification is possible. Instead, the recombinant DNA integrates into the genome of the engineered cell, where the promoter directs expression of the desired nucleic acid.

Examples of mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from

Epstein Bar virus include pHEBO, and p205. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Recombinant viral vectors may also be used to synthesize the polynucleotides of the present invention. Viruses are very specialized infectious agents that have evolved, in many cases, to elude host defense mechanisms. Typically, viruses infect and propagate in specific cell types. The targeting specificity of viral vectors utilizes its natural specificity to specifically target predetermined cell types and thereby introduce a recombinant gene into the infected cell. Bone marrow cells can be targeted using the human T cell leukemia virus type I (HTLV-I).

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptide variants of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Introduction of nucleic acids by viral infection offers several advantages over other methods such as lipofection and electroporation, since higher transfection efficiency can be obtained due to the infectious nature of viruses.

As mentioned, the agents may be the polypeptides themselves. The polypeptides may be recombinant polypeptides.

The present invention contemplates additional agents upstream of the hnRNP A/B pathway which act to increase the amount and/or activity of this polypeptide in the brain.

It will be appreciated that the amount and activity of hnRNP A/B is regulated on various levels, including on the gene level (e.g. transcription) and the protein level (e.g. translation, stability, localization). The present invention therefore contemplates any agents which affects at least one of these parameters.

As described in the Examples section below, the present inventors have shown that the amount of hnRNP A/B is also regulated on the miRNA level. Specifically, the present inventors have shown that there is an increased expression of miRNAs which target (and therefore down-regulate) hnRNPA/B in brain samples of Alzheimer's patients compared with control patients miRNAs (FIGS. 7G-H). Thus, the present invention contemplates agents which decrease the amount of miRNAs that target hnRNPA/B.

One such miRNA that was shown to target and downregulate hnRNP A/B is miR211 (SEQ ID NO: 91). Thus, the present invention contemplates antagonists of miR211. Such antagonists may be single stranded oligonucleotides which hybridize under physiological conditions with miR211 (e.g. SEQ ID NO: 87). Alternatively, the antagonists may be additional miRNAs which work up or downstream of miR211, bringing about their down-regulation. An example of such a miRNA is miRNA 132 (SEQ ID NO: 86).

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms and have been shown to play a role in development, homeostasis, and disease etiology.

Below is a brief description of the mechanism of miRNA activity.

Genes coding for miRNAs are transcribed leading to production of an miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nt precursor known as the pre-miRNA. Drosha cleaves the pri-miRNA with a staggered cut typical of RNase III endonucleases yielding a pre-miRNA stem loop with a 5′ phosphate and ˜2 nucleotide 3′ overhang. It is estimated that approximately one helical turn of stem (˜10 nucleotides) extending beyond the Drosha cleavage site is essential for efficient processing. The pre-miRNA is then actively transported from the nucleus to the cytoplasm by Ran-GTP and the export receptor exportin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. miRNA* sequences may be found in libraries of cloned miRNAs but typically at lower frequency than the miRNAs.

Although initially present as a double-stranded species with miRNA*, the miRNA eventually become incorporated as a single-stranded RNA into a ribonucleoprotein complex known as the RNA-induced silencing complex (RISC). Various proteins can form the RISC, which can lead to variability in specifity for to miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 Genes Dev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

MiRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

The term “microRNA mimic” refers to synthetic non-coding RNAs that are capable of entering the RNAi pathway and regulating gene expression. miRNA mimics imitate the function of endogenous microRNAs (miRNAs) and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O,4′-C-ethylene-bridged nucleic acids (ENA)). For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA.

It will be appreciated from the description provided herein above, that administering a miRNA agent may be affected in a number of ways:
1. Transiently transfecting cells with the mature double stranded miRNA or single stranded miRNA antagonist;
2. Stably, or transiently transfecting stem cells with an expression vector which encodes the mature miRNA.
3. Stably, or transiently transfecting the cells with an expression vector which encodes the pre-miRNA. The pre-miRNA sequence may comprise from 45-90, 60-80 or 60-70 nucleotides. The sequence of the pre-miRNA may comprise a miRNA and a miRNA* as set forth herein. The sequence of the pre-miRNA may also be that of a pri-miRNA excluding from 0-160 nucleotides from the 5′ and 3′ ends of the pri-miRNA. The sequence of the pre-miRNA may comprise the sequence of the miRNA—i.e. SEQ ID NO: 86 or variants thereof.
4. Stably, or transiently transfecting cells with an expression vector which encodes the pri-miRNA. The pri-miRNA sequence may comprise from 45-30,000, 50-25,000, 100-20,000, 1,000-1,500 or 80-100 nucleotides. The sequence of the pri-miRNA may comprise a pre-miRNA, miRNA and miRNA*, as set forth herein, and variants thereof. Preparation of miRNAs mimics can be effected by chemical synthesis methods or by recombinant methods.

It will be appreciated that the polynucleotide agents of some embodiments of the invention can also utilize homologues which exhibit the desired activity (i.e., down-regulation of hnRNP A/B). Such homologues can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to any of the sequences SEQ ID NOs: 86 or 87, as determined using the BestFit software of the Wisconsin sequence analysis package, utilizing the Smith and Waterman algorithm, where gap weight equals 50, length weight equals 3, average match equals 10 and average mismatch equals −9.

It will be appreciated that the cells can be treated in vivo (i.e., inside the organism or the subject) or ex vivo (e.g., in a tissue culture). In case the cells are treated ex vivo, the method preferably includes a step of administering such cells back to the individual (ex vivo cell therapy).

Administration of the ex vivo treated cells of the present invention can be effected using any suitable route of introduction, such as intravenous, intraperitoneal, intra-kidney, intra-gastrointestinal track, subcutaneous, transcutaneous, intramuscular, intracutaneous, intrathecal, epidural, and rectal. According to presently preferred embodiments, the ex vivo treated cells of the present invention may be introduced directly into the brain of the subject.

The cells used for ex vivo treatment according to the present invention can be to derived from either autologous sources, or from allogeneic sources, such as bone marrow or other cells derived from non-autologous sources. Since non-autologous cells are likely to induce an immune reaction when administered to the body, several approaches have been developed to reduce the likelihood of rejection of non-autologous cells. These include either suppressing the recipient immune system or encapsulating the non-autologous cells or tissues in immunoisolating, semipermeable membranes before transplantation.

The agent, the polynucleotide and/or the expression vector of the present invention can be administered to the individual per se or as part of a pharmaceutical composition where it is mixed with suitable carriers or excipients.

As used herein, a “pharmaceutical composition” refers to a preparation of one or more of the active ingredients described herein with other chemical components such as physiologically suitable carriers and excipients. The purpose of a pharmaceutical composition is to facilitate administration of a compound to an organism.

As used herein, the term “active ingredient” refers to the agent, the polynucleotide and/or the expression vector of the present invention accountable for the intended biological effect.

Hereinafter, the phrases “physiologically acceptable carrier” and “pharmaceutically acceptable carrier,” which may be used interchangeably, refer to a carrier or a diluent that does not cause significant irritation to an organism and does not abrogate the biological activity and properties of the administered compound. An adjuvant is included under these phrases.

Herein, the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils, and polyethylene glycols.

Techniques for formulation and administration of drugs may be found in the latest edition of “Remington's Pharmaceutical Sciences,” Mack Publishing Co., Easton, Pa., which is herein fully incorporated by reference.

Suitable routes of administration may, for example, include oral, rectal, transmucosal, especially transnasal, intestinal, or parenteral delivery, including intramuscular, subcutaneous, and intramedullary injections, as well as intrathecal, direct intraventricular, intravenous, inrtaperitoneal, intracardiac, intranasal, or intraocular injections.

Alternately, one may administer the pharmaceutical composition in a local rather than systemic manner, for example, via injection of the pharmaceutical composition directly into a tissue region (e.g. brain) of a patient.

According to a particular embodiment, the pharmaceutical composition is formulated for crossing the blood brain bather.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers, such as lipophilic tails, e.g. long chain alkyl, which enhance penetration or activity of the IDE inhibitors); and the transitory disruption of the integrity of the BBB by hyperosmotic disruption (resulting from the infusion of a mannitol solution into the carotid artery or the use of a biologically active agent such as an angiotensin peptide).

Methods for drug delivery behind the BBB include intracerebral implantation (such as with needles) and convection-enhanced distribution. Mannitol can be used in bypassing the BBB. Likewise, mucosal (e.g., nasal) administration can be used to bypass the BBB.

Pharmaceutical compositions of the present invention may be manufactured by processes well known in the art, e.g., by means of conventional mixing, dissolving, granulating, dragee-making, levigating, emulsifying, encapsulating, entrapping, or lyophilizing processes.

Pharmaceutical compositions for use in accordance with the present invention thus may be formulated in conventional manner using one or more physiologically acceptable carriers comprising excipients and auxiliaries, which facilitate processing of to the active ingredients into preparations that can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries as desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, and sodium carbomethylcellulose; and/or physiologically acceptable polymers such as polyvinylpyrrolidone (PVP). If desired, disintegrating agents, such as cross-linked polyvinyl pyrrolidone, agar, or alginic acid or a salt thereof, such as sodium alginate, may be added.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions that can be used orally include push-fit capsules made of gelatin, as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate, and, optionally, stabilizers. In soft capsules, the active ingredients to may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane, or carbon dioxide. In the case of a pressurized aerosol, the dosage may be determined by providing a valve to deliver a metered amount. Capsules and cartridges of, for example, gelatin for use in a dispenser may be formulated containing a powder mix of the compound and a suitable powder base, such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with, optionally, an added preservative. The compositions may be suspensions, solutions, or emulsions in oily or aqueous vehicles, and may contain formulatory agents such as suspending, stabilizing, and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acid esters such as ethyl oleate, triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the active ingredients, to allow for the preparation of highly concentrated solutions.

Alternatively, the active ingredient may be in powder form for constitution with a suitable vehicle, e.g., a sterile, pyrogen-free, water-based solution, before use.

The pharmaceutical composition of the present invention may also be formulated in rectal compositions such as suppositories or retention enemas, using, for example, conventional suppository bases such as cocoa butter or other glycerides.

Pharmaceutical compositions suitable for use in the context of the present invention include compositions wherein the active ingredients are contained in an amount effective to achieve the intended purpose. More specifically, a “therapeutically effective amount” means an amount of active ingredients (e.g., the agent, the polynucleotide and/or the expression vector of the present invention) effective to prevent, alleviate, or ameliorate symptoms of the pathology [e.g., a pathology related to an AChE-associated biological pathway such as thrombocytopenia, idiopathic thrombocytopenic purpura (ITP), congenital amegakaryocytic thrombocytopenia (CAMT), essential thrombocythemia (ET), acquired amegakaryocytic thrombocytopenia (AATP)] or prolong the survival of the subject being treated.

Determination of a therapeutically effective amount is well within the capability of those skilled in the art, especially in light of the detailed disclosure provided herein.

For any preparation used in the methods of the invention, the dosage or the therapeutically effective amount can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration, and dosage can be chosen by the individual physician in view of the patient's condition. (See, e.g., Fingl, E. et al. (1975), “The Pharmacological Basis of Therapeutics,” Ch. 1, p. 1.)

Dosage amount and administration intervals may be adjusted individually to provide sufficient plasma or brain levels of the active ingredient to induce or suppress the biological effect (i.e., minimally effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks, or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA-approved kit, which may contain one or more unit dosage forms containing the active ingredient. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising a preparation of the invention formulated in a pharmaceutically acceptable carrier may also be prepared, placed in an appropriate container, and labeled for treatment of an indicated condition, as further detailed above.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Cholinergic-Mediated microRNA Control of hnRNPs A/B Regulates Cortical Alternative Pplicing and Neural Functioning

Materials and Methods

Human samples: Post mortem cortical samples of AD patients and controls were obtained.

Transgenic Mice: APPsw/PS 1ΔE9 mice expressing both transgenes under mouse prion protein promoter were purchased from Jackson Laboratory (Bar Harbor, Me.). Mice were maintained as a hemizygote by crossing transgenics to animals on a B6C3F1/J background. Tail-tip PCR was used for genotyping. Brain sections of Tau mice expressing double mutated K257T/P301S Tau from its natural promoter samples and age-matched controls were as described in Rosenmann et al., 2008 [Exp Neurol 212, 71-84].

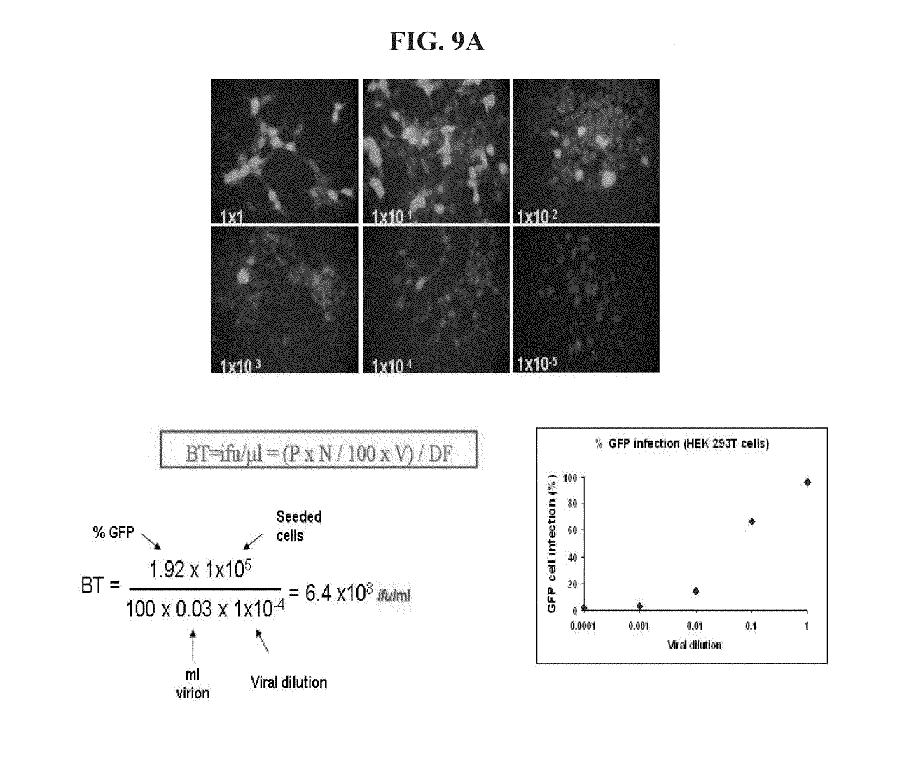

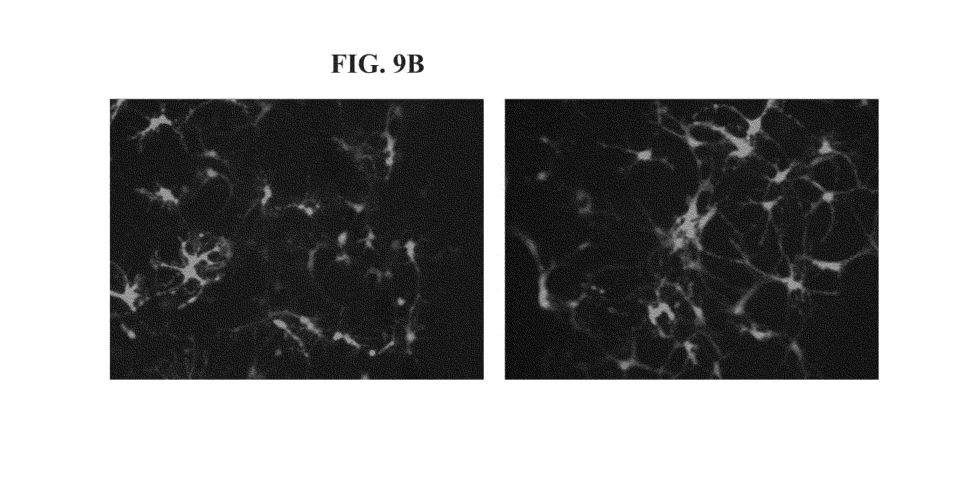

Lentivirus-encoded shRNAs: Active viral particles were produced by co-transfecting OpenBiosystem's TRC pre-cloned shRNA in pLKO.1-Puro vector with plasmids coding for delta R8.2 and VSV-G into HEK-293T cells, using polyethylenimine (Sigma). The packaged virus was collected at 24 and 48 h post transfection and concentrated using ultracentrifugation (70,000 g, 2 h, 15° C.). Dilutions of concentrated virus were followed by infection of HEK-293T cells with diluted virus. The resulting titer was assessed for shRNA expressing viruses using puromycin selection and for GFP expressing viruses counting fluorescing cells (See FIG. 9-B).

ECoG: Electrocorticography (ECoG) recordings were performed using a subcutaneously implanted bipolar transmitter (TA10EA-F20, Data Science International, St. Paul, Minn.) attached to a pure iridium electrode (WPI, Berlin, Germany) that was perpendicularly inserted in the coordinates described in the EC stereotactic injections (length of the electrode according to the DV coordinate). Reference electrode was placed above the dura through a hole drilled in the skull located 1.5 mm posterior and 1.5 mm lateral to bregma. Both electrodes were affixed with bone-cement (Unifast Trad, GC America, Alsip, Ill.). Antibiotic treatment (5 mg/kg p.o. enrofloxacin, Bayer, Germany) was given during recovery (7 days). ECoG recordings (at 0.5 or 1 kHz) were performed from 7 days post-operation. All 1 kHz were changed off-line to 0.5 Hz sampling rate. The recording system includes a channel that indicates the quality of the signal for each sample, termed “activity channel”. Signal fluctuations of the activity channel indicate movements made by the animal. The activity channel output was used to define the level of motor activity of the animal. Minuets of ECoG recordings were defined as “sleep epochs” if there was a flat signal in the activity channel, and hence no motor activity of the animal, in the examined minute and 5 minutes back in time. “Wakefulness epochs” were defined as minutes where robust fluctuations, above a certain threshold, were visible in activity channel. Fourier transform was performed to get the power spectrum of each minute and then averaged all minuets for each animal in the frequency space. Power spectrums for 3 mice of each group were averaged and statistically analyzed.

Brain specimens: Mice were anesthetized by isoflurane inhalation and decapitated. One hemisphere was transformed to fresh 4% paraformaldehyde in PBS solution immediately after dissection and later embedded in paraffin for histological analysis. The other hemisphere was dissected further, frozen in liquid nitrogen and stored at −70° C. for subsequent analyses.

Primary mixed cortical cultures: Primary mouse cultures of neurons and glia were produced from E16 embryos. Pregnant mice were anesthetized with isoflurane and sacrificed by cervical dislocation. The uterus was removed and placed on ice. After 5 minutes the heads were dissected under binocular and cortices were removed, placed in cold DMEM, and triturated with a scalpel and up and down pippeting. The medium was replaced with neurobasal medium supplemented with 1:50 B27, 2 mM L-Glutamine (Invitrogen, Grand Island, N.Y.), 50 U penicillin and 50 μg/ml streptomycin (Biological Industries, Beit-haemek, Israel), cells were counted and plated in 12 or 24-well plated pre-treated with 10 μg/ml poly-lysine (Sigma, St. Louis). For confocal microscopy cells were plated on glass cover slips similarly treated.

RNA isolation and RT PCR: Tissues or cells were homogenized in tri reagent (Sigma) or miRNeasy (Qiagen) and total RNA was extracted as per the manufacturer's protocol. RNA concentration was determined with Nanodrop (Thermo, Wilmington, Del.) and RNA quality was assessed by gel-electrophoresis. For human samples used in the exon array analysis, RNA quality was determined with Bioanalyzer (Agilent, Santa Clara, Calif.). RIN values of CT samples: 5.9, 7.4, 7.1; of AD patients: 6.1, 5.9, 5.6. These samples were thus validated not to be degraded. cDNA synthesis (Promega, Madison, WI) involved 0.4 μg RNA samples in 20 μl reactions. Duplicate real-time reverse transcriptase (RT)-PCR tests involved ABI prism 7900HT, SYBR green master mix (Applied biosystems, Foster City, Calif.) and ROX, a passive reference dye for signal normalization across the plate. For agarose gel analysis, PCR was performed using Taq DNA polymerase (Sigma). Primer sequences are listed in Table 1, herein below.

TABLE 1
Real-time primers
Human primers

| | | |
|---|---|---|
| enah_I5+ | TTCTCCCTTCTCCCTCTGC - | SEQ ID NO: 1 |
| enah_I5− | GTGGTCCCAAGACAATGC | SEQ ID NO: 2 |
| enah_E4/5+ | CCTGCCTCTGTTGAGACTCC | SEQ ID NO: 3 |
| dystonin_I95+ | TTCTGCTAACAGTATTCTTTAATGTGA | SEQ ID NO: 4 |
| dystonin_I95− | TGAAGAGTTTGATTCCGAACG | SEQ ID NO: 5 |
| dystonin_E96− | AGCCAGGGTATGGCTGCT | SEQ ID NO: 6 |
| dystonin_E96+ | CAGCCTGACTGGACACAGAA | SEQ ID NO: 7 |
| dystonin_I40− | ATCTACTGTGCCCAGCGACT | SEQ ID NO: 8 |
| dystonin_I40+ | GAAAGGATCATCTCTACTTTCTGG | SEQ ID NO: 9 |
| hena_E4+ | GTGGCGAGATGCTAGACAG | SEQ ID NO: 10 |
| hena_E4− | ATTTTGAACTTGAGCAGGTAGTTG | SEQ ID NO: 11 |
| hena_E5/6+ | GGGAGAGAGAGCGCAGAATA | SEQ ID NO: 12 |
| hena_E5/6− | AAGCCTGGCTCAGAAGCA | SEQ ID NO: 13 |
| RELN_E44+ | TCATGAGACTGGGATGTGGT | SEQ ID NO: 14 |
| RELN_E44− | TGAAGAAGACTCCACGAGAGG | SEQ ID NO: 15 |
| RELN_E43+ | GTGGGAAACCATCTCGAAAG | SEQ ID NO: 16 |
| RELN_E43− | AAATCCAGGTCTCGTGTCATC | SEQ ID NO: 17 |
| RELN_E41+ | GAGACTGGAATTTTCAAGGGACT | SEQ ID NO: 18 |
| RELN_E41− | CTCGGTGGAGCATAAAGAGC | SEQ ID NO: 19 |
| RELN_E38+ | TCTCCATCAGTGGAGGAATCA | SEQ ID NO: 20 |
| RELN_E38− | GCAGTGTATGGCAAGGGAAC | SEQ ID NO: 21 |
| RELN_E35+ | ACTACACTGTGGGGGCTGAT | SEQ ID NO: 22 |
| RELN_E35− | GGATTTGTGATGCTGGACG | SEQ ID NO: 23 |
| RELN_E30+ | TAGGTTTGAGGGGAAGCTCA | SEQ ID NO: 24 |
| RELN_E30− | GAAGTAGAGAGATTTGCCATCG | SEQ ID NO: 25 |
| RELN_E20+ | ATCATGACATCTGTGCTTTTCAA | SEQ ID NO: 26 |
| RELN_E20− | ATGGCTGAACATTTCCAAGG | SEQ ID NO: 27 |
| RELN_E18+ | TGTGACCCTGGATTTTCTGG | SEQ ID NO: 28 |
| RELN_E18− | AAAACCAGGGCCTTACCACT | SEQ ID NO: 29 |
| RELN_E15+ | GCTTGGAATTTTCTACCAACCA | SEQ ID NO: 30 |
| RELN_E15− | AGAGGAGTAGACAGTGCTGTGG | SEQ ID NO: 31 |
| RELN_E11+ | TCAGTCAGATGGGAACTCCA | SEQ ID NO: 32 |
| RELN_E11− | TTCTTCTGACCATTGCTCTTGA | SEQ ID NO: 33 |
| RELN_E5/6+ | CTCCAACAGATGTCACTGTGC | SEQ ID NO: 34 |
| RELN_E5/6− | GGATTTAATTGCAGTTGGTGGT | SEQ ID NO: 35 |
| RELN_E3+ | GGATCATGTCTGACCACCAG | SEQ ID NO: 36 |
| RELN_E3− | AGGTGGAGCAATCCAGATGA | SEQ ID NO: 37 |
| RELN_E13/14+ | GAGGAATTTGTGACCCTGGA | SEQ ID NO: 38 |
| RELN_E13/14+ | TTGATGGTTCTTTTGCCTGA | SEQ ID NO: 39 |
| RELN_E24/25+ | AAACCAGAACAAGGGTGTGG | SEQ ID NO: 40 |
| RELN_E24/25− | CTCTGCTGTCAGGCTTGTTG | SEQ ID NO: 41 |
| DRAM2 E2+ | CGGAGAAAATCAGCGGTCTA | SEQ ID NO: 42 |
| DRAM2 E2− | CGTGTACTCAACAGGAACGTG | SEQ ID NO: 43 |
| DRAM2 E4/5+ | ACTGCAGTAACACTCCACCAT | SEQ ID NO: 44 |
| DRAM2 E4/5− | AAACTGCCGCAATATTTAGCAT | SEQ ID NO: 45 |
| CD55 E7/8+ | TCCAACTACAGAAGTCTCACCAA | SEQ ID NO: 46 |
| CD55 E7/8− | TGGTTGTCCTGGAAACAGGT | SEQ ID NO: 47 |
| CD55 E5/6+ | GCAGCTCTGTCCAGTGGAGT | SEQ ID NO: 48 |
| CD55 E5/6− | GGTCACGTTCCCCTTGAAT | SEQ ID NO: 49 |
| SIPA1L1_E14/15+ | GGCAGAACACCCAGTCAGAT | SEQ ID NO: 50 |
| SIPA1L1_E14/15− | ATGCTGCTCGAGGACAAAA | SEQ ID NO: 51 |
| SIPA1L1_E4/5+ | TTTTGGGGCTGATGAGAATC | SEQ ID NO: 52 |
| SIPA1L1_E4/5− | GGACCGAACCTCTCAGTGTC | SEQ ID NO: 53 |

mouse primers

| | | |
|---|---|---|
| mCD55E8+ | CCCAGCATGTACCTGTTACC | SEQ ID NO: 54 |
| mCD55E8− | TCACATGCAAAACTGTCAAGG | SEQ ID NO: 55 |
| mCD55E83ss+ | GCCACAGCAAAACCTTCATT | SEQ ID NO: 56 |
| mCD55E83ss− | AACAGGTACATGCTGGGTTG | SEQ ID NO: 57 |
| mCD55E1/2+ | TGTCTCTGTTGCTGCTGTCC | SEQ ID NO: 58 |
| mCD55E1/2− | TGCTCAGCAAACTTGGAGTG | SEQ ID NO: 59 |
| mDRAM2E2+ | TGATTCAAGGTTCACACTCACA | SEQ ID NO: 60 |
| mDRAM2E2− | AAAACTGAGGCCTTGCTGAA | SEQ ID NO: 61 |
| mDRAM2E3/4+ | TTCAGCAAGGCCTCAGTTTT | SEQ ID NO: 62 |
| mDRAM2E3/4− | TCAGGAGGTATTGTCCCTGTG | SEQ ID NO: 63 |
| mMena+ | CGGCAGTAAGTCACCTGTCA | SEQ ID NO: 64 |
| mMena− | CTTCAGCTTTGCCAGCTCTT | SEQ ID NO: 65 |
| mMenaINV2/3+ | GATTCAAGACCATCAGGTTGTG | SEQ ID NO: 66 |
| mMenaINV2− | CAATGTTGGCCCTAAATAGAA | SEQ ID NO: 67 |
| mSIPA1L1_I5+ | TCAGGCATGCAGTTCTTTTG | SEQ ID NO: 68 |
| mSIPA1L1_I5− | GAAAGCAGGCAGTACCTTCG | SEQ ID NO: 69 |
| mSIPA1L1_E4/5+ | TAGTGTGGACGCTGCTGTCT | SEQ ID NO: 70 |
| mSIPA1L1_E4/5− | GGCTCTGTGGTCACCAGAAT | SEQ ID NO: 71 |
| mDST E41+ | ATGGCATTTCCCCCATTAG | SEQ ID NO: 72 |
| mDST E41− | GGAGGTTGGTTTTGCTTCAA | SEQ ID NO: 73 |
| mDST E7/8+ | GAGCGGGACAAAGTTCAAAA | SEQ ID NO: 74 |
| mDST E7/8− | CCCGTCCCTCAGATCCTC | SEQ ID NO: 75 |
| mRELN E3+ | ATCATGTCCGACCACCAGTT | SEQ ID NO: 76 |
| mRELN E3− | GGCAATCCAGACAAAGCTGA | SEQ ID NO: 77 |
| mRELN E18+ | GCAGTGCCAGACTTTCCTCT | SEQ ID NO: 78 |
| mRELN E18− | GCCTCCCATCTTTGTTGAAA | SEQ ID NO: 79 |
| mRELN E1/2+ | GGCAACCCCACCTACTACG | SEQ ID NO: 80 |
| mRELN E1/2− | GACTGGATGCTTGTCGAGGT | SEQ ID NO: 81 |

primers for gel analysis

| | | |
|---|---|---|
| CD55+ | AGGTCCCACCAACAGTTCAG | SEQ ID NO: 82 |
| CD55− | CGAGACTGCAGTGAGCTACG | SEQ ID NO: 83 |
| Dram2+ | GCGCTAGTCGGTCTGGTAAG | SEQ ID NO: 84 |
| Dram2− | AAACTGAGGCCTTGCTGAAA | SEQ ID NO: 85 |

β-actin and GAPDH were used as reference transcripts. Annealing temperature was 60° C. for all primers. Serial dilution of samples served to evaluate primers efficiency and the appropriate cDNA concentration that yields linear changes. -RT controls verified lack of genomic DNA. miRNA quantification using The TaqMan miRNA Assays (Applied Biosystems) was conducted in two-step RT-PCR kit according to the manufacturer's instructions.

Immunohistochemistry and immunofluorescence: Paraffin slides were rehydrated by washing in xylene and serial dilutions of ethanol in water. Heat-induced antigen retrieval involved boiling slides in 10 mM pH 6 citrate buffer for 10 minutes. Hydrogen peroxide methanol quench was performed for slides later developed with 3,3′-Diaminobenzidine tetrahydrochloride (DAB). After washing, slides were incubated with 150 μL/slide of blocking buffer (4% normal serum, 0.05% tween20, 0.3% triton X-100) for 60 minutes, followed by over-night incubation at 4° C. with primary antibody diluted in the blocking buffer. Slides were then washed and incubated with biotin-conjugated secondary antibody for 2 hours, after which detection was performed by streptavidin-conjugated Cy (Jackson, West Grove, Pa.) or by horse reddish peroxidase (HRP) and DAB using the ABC elite kit (Vector, Burlingame, Calif.). Nuclear staining with 4′,6-diamidino-2-phenylindole (DAPI) was used as counter-staining. Antibodies used include: anti hnRNP A/B (Hua et al., 2008), hnRNP A1, hnRNP A2/B1, hnRNP A3, actin, neuroligin 3 (Santa Cruz, Santa Cruz, Calif.), activated caspase 9, Synaptophysin, MAP2 (Cell Signaling, Danvers, Mass.), SR proteins (Invitrogen), human APP (Covance, Emeryville, Calif.) and GFAP (Sigma). Antibodies dilution: 1:1000 for Western blots, and 1:200 for immunohistochemistry and immunofluorescence.

Stereotactic injections: Nine weeks old male C57B1/6J mice were group housed until they underwent stereotaxic surgery, after which they were singly-housed throughout all subsequent testing, at a constant temperature (22±1° C.) and 12-h light/dark cycles. Mice were anesthetized by i.p. injections of ketamine (50 mg/kg) (Forth Dodge, Iowa, USA) and domitor (0.5 mg/kg) (Orion Pharma, Espoo, Finland) mix, and then mounted in a stereotaxic apparatus for intrahippocampal injections. 0.4 μg/ul mu-P75 SAP (advanced targeting systems, San Diego, Calif.) was injected intracerebroventricularly (ICV) at the following coordinates (in mm) relative to bregma: were as follows: AP: −2.0, L: ±1.8, DV: −1.5. Lenti viruses encoding shRNA agents were injected into the entorhinal cortex at: AP: −3.6, L: ±6.8, DV: −4.5. Bilateral injections of 1 μl were conducted using a 10 μl Glenco syringe (Huston, Tex., USA). After each injection, the needle was left in situ for 5 min before being slowly retracted to allow complete diffusion.

Behavioral analysis: Rotarod test: An accelerating rotarod (Ugo Basile, Comerio VA, Italy) was used for testing motor coordination. The rotarod was set to accelerate from 4 to 40 rpm over 5 minutes. On the first day, mice were placed on the rotating drum until they did not fall for a period of 30 s. Mice were then tested in 3 trials with a minimum of 2 minutes elapsed between trials.

Sensory-motor function tests: Mice were examined at the peak of their motor activity during the dark phase. Deficits in vestibular function were tested by holding mice by the tail, lifting and then lowering them over a metal cage top. Then, while the mouse was still, a tactile stimulus to the right or left shoulder and trunk was applied using a cotton swab, and orienting to the stimulus was noted. Efficient and symmetric head and limb placement, and symmetric head orienting to tactile stimuli were taken as evidence of normal vestibular functioning.

Water maze test: The water maze consisted of a round tank, 1.6 m in diameter, filled with water. Mice were trained to find the location of a hidden platform (16 cm in diameter), submerged 1 cm below the water surface, using extra maze visual cues. The training part consisted of 4 trials per day, with a 1-h brake between trials, for 3 days. The escape latency, i.e. the time required by the mouse to find the platform and climb it, was recorded for up to 60 s. Each mouse was allowed to remain on the platform for 30 sec and was then moved from the maze to its home cage. If the mouse did not find the platform within 60 sec, it was placed gently on the platform for 30 sec, and then returned to its home cage. On the 4^{th} day of the experiment, the platform was removed and a probe trial was conducted: mice were placed in the maze for 60 sec, in which the number of crosses over the four quadrants of the maze was recorded. Increased swimming in the quadrant where the platform was originally placed was considered as an indication of spatial acquisition.

Exon array: 1 μg of total RNA from 3 non-demented female cotrols and 3 female AD patients (averge age 73 and 76.5 respectively) was labeled with the Affymetrix exon array whole transcripts sense targeting labeling assay and reagents, including r-RNA reduction and labeling with Streptavidin-phycoerithrin. Each sample was hybridized to a GeneChip® Exon 1.0 ST Array (Affymetrix, Santa Clara, Calif., USA) according to manufacturer's instructions, and results were scanned to create .CEL files using Affymetrix GCS 3000 7G scanner and GeneChip Operating Software v. 1.3 to produce CEL intensity files. Array analysis was performed with Partek Genomic suite (Partek, St. Louis, Mo.) and Altanalyze (Emig et al., 2010). Array data is available from NCBI's Gene expression omnibus (GEO), Series record GSE26972.

Statistical analysis: All analysis was done using Prism 5 software. Statistical significance was calculated using Mann-Whitney test, Student's t-test or by one- or two-way ANOVA with LSD post-hoc, where appropriate. Unless otherwise noted, % from control average ±SEM is shown for all graphs.

Results

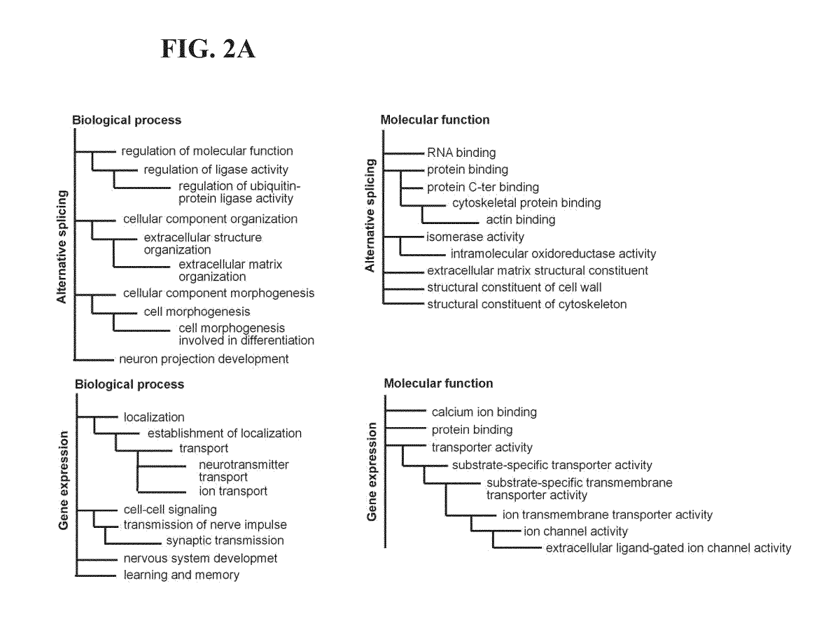

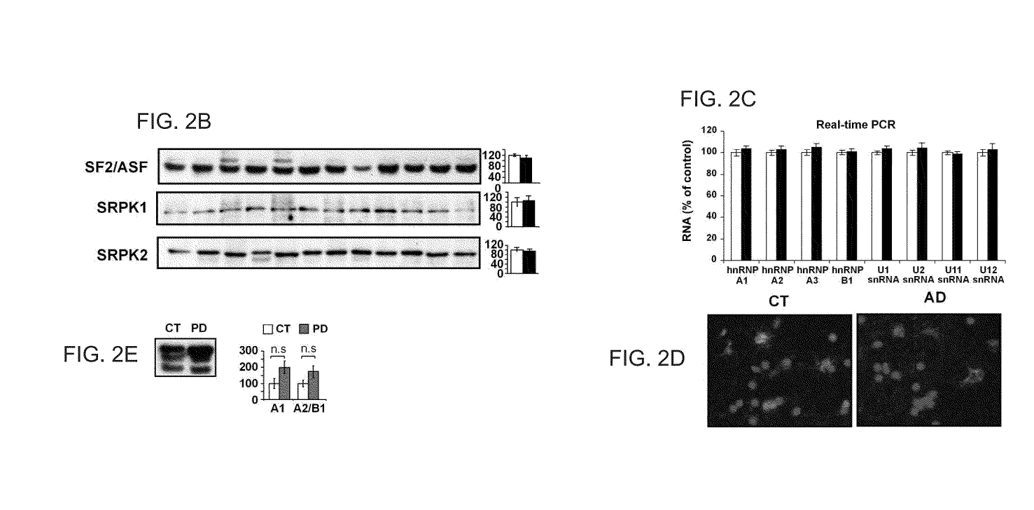

For initiating an unbiased search for transcriptional changes in AD, exon-level microarray analysis on post-mortem entorhinal cortices from AD patients and age-matched non-demented controls was applied. To gain focused information on alternative splicing, the expression level for each known exon in the human transcriptome relative to the expression of the entire gene was calculated, and transcripts whose alternative splicing patterns but not general transcript levels were modified were identified, thus gaining access to previously unexplored transcript groups. 531 and 407 genes were under- or over-expressed by more than 2-fold in AD samples, respectively (FIG. 1A). These belong to multiple gene ontology (GO) categories related to synaptic transmission (FIG. 2A), deficits which are characteristic of AD. In comparison, 383 alternative splicing events in 319 genes were identified, including GO categories such as regulation of ubiquitin protein-ligase activity, isomerase activity, neuron projections and RNA-binding activity (FIG. 2A). Thus, splicing and transcription modifications both affected large gene groups of neurodegeneration-relevant categories. As opposed to neuronal excitation which has been shown to increase exon exclusion events, 75% of the AD-alternatively spliced to exons demonstrated increased inclusion, predicting an increase in splicing enhancers or reductions in splicing suppressors (FIG. 1B). In a larger set of human samples (n=7 in each group), reverse transcribed polymerase-chain reaction (RT-PCR) and real-time RT-PCR, validated alternative splicing events in transcripts involved in dendritic spine morphology (SIPA1L1,(Pak et al., 2001)), synaptic plasticity (Reelin, (Kocherhans et al.)), neurodegeneration (Dystonin, (Sonnenberg and Liem, 2007)), neuronal response to injury (CD55, (Wang et al.)) and cell death (DRAM2, (Park et al., 2009)) (FIG. 1C,D).

In splice site selection, the serine/arginine proteins (SR proteins) bind to splicing enhancer elements whereas heterogeneous nuclear ribonucleoproteins (hnRNPs) bind to silencer elements, and inhibit the splicing reaction. The present inventors therefore analyzed protein and mRNA levels of several splicing factors and spliceosomal components in the entorhinal cortex of AD patients and controls. No changes in SR protein levels (FIG. 1G), the SR kinases SRPK1 and SRPK2 or small nuclear RNA (U1, U2, U11, U12) were observed (FIGS. 2B-C). In contrast, hnRNP A/B were almost completely absent in entorhinal cortices from AD patients, unlike the robust nuclear staining of control tissues (FIG. 1E). These immunohistochemistry results were validated by immuno-blotting (FIG. 1F) using pan-hnRNP A/B monoclonal antibody and antibodies selective for A1, A2/B1 and the minor hnRNP A3. In contrast, post-mortem substantia nigra pars compacta samples from Parkinson's disease (PD) patients showed no reduction of hnRNP A/B compared to controls (FIG. 2E), indicating that general neuronal death does not account for hnRNP A/B loss and may be AD-specific. Furthermore, hnRNP A/B mRNA levels were not altered in AD, implicating the involvement of post-transcriptional mechanisms.

To explore the functional consequences of the hnRNP A/B decline observed in AD, mouse primary mixed cortical cultures were transduced with lenti-virus particles encoding short-hairpin RNA directed against hnRNP A1 or A2/B1 (here termed shRNA A1 and shRNA A2, respectively). An shRNA sequence that is not predicted to bind any target in the mouse (shRNAct) served as control.

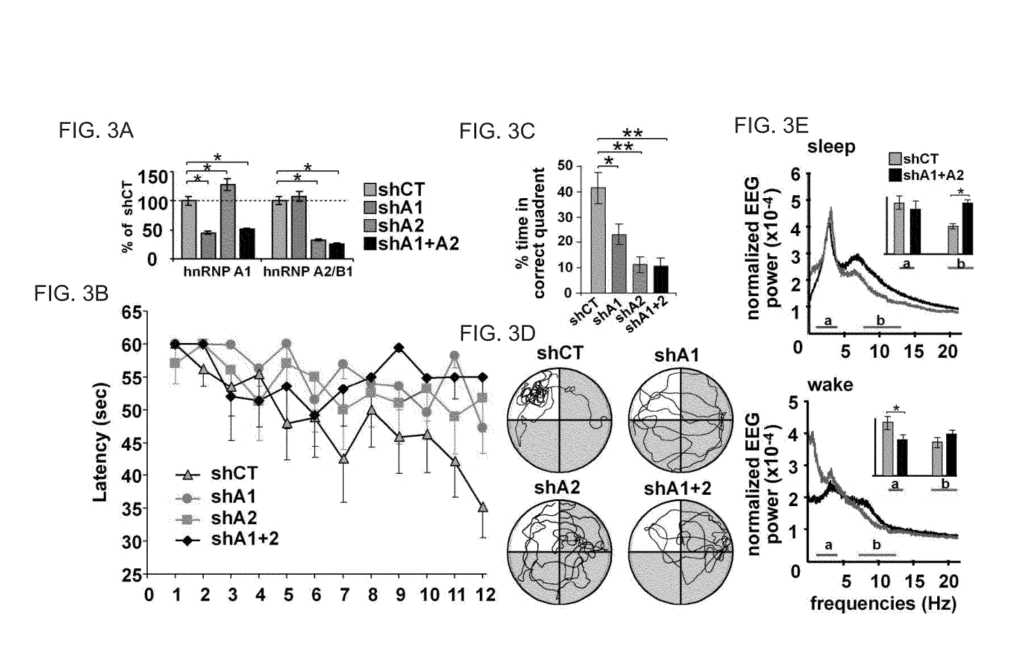

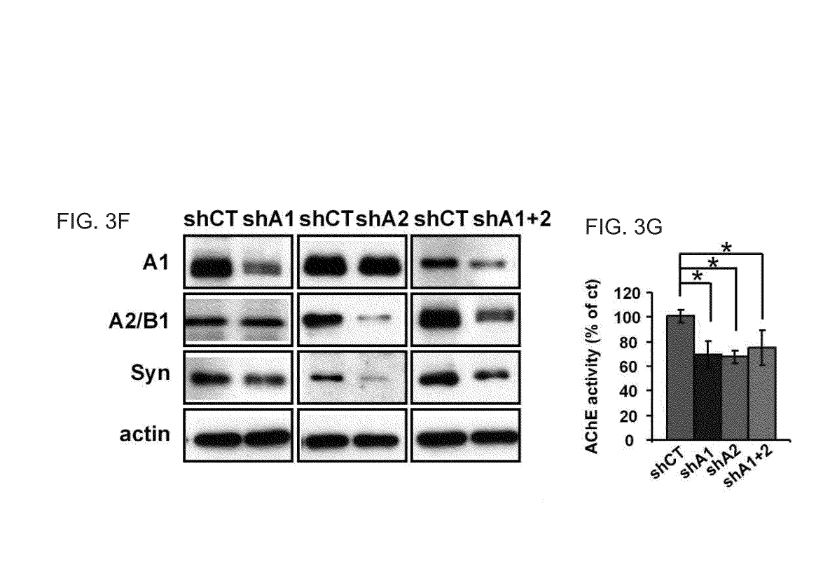

Knocking-down hnRNP A1 did not reduce A2/B1 mRNA levels and vice versa (FIG. 3A), demonstrating selectivity and specificity. However, a reduction of hnRNP to A1 resulted in a minor (˜20%) but significant increase of hnRNP A2/B1, which may suggest a compensatory feedback response. To exclude any residual redundancy, the present inventors therefore added to all consequent experiments a double knock-down of hnRNP A1 and hnRNP A2/B1.

The present inventors efficient knock-down in primary neurons at both the mRNA (data not shown) and protein levels (FIG. 3F), compared to an shRNA sequence that is not predicted to bind any target in the mouse transcriptome (shRNAct). Acetylcholinesterase activity in the culture medium was also significantly reduced, mimicking yet another hallmark of AD (FIG. 3G).

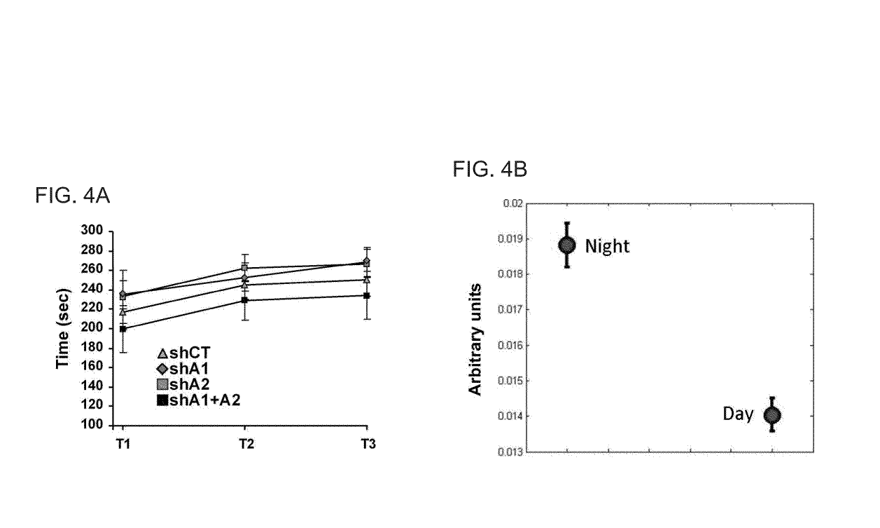

Next, the present inventors asked if hnRNP A/B reduction in-vivo impairs learning and memory, the major phenotypic characterization of AD patients. One month following stereotaxic injection of shRNA viruses to the entorhinal cortex of C57/B6 mice, mice were subjected to sensory-motor tests and any possible deficit in their motor activity or visual capabilities was excluded (FIGS. 4A-B). In the Morris water maze, shRNA A1, shRNA A2 and shRNA A1+A2-injected mice showed significantly longer latency to reach a hidden platform than shRNAct injected mice (FIG. 3B), suggesting impaired learning and memory capabilities. In addition, when the platform was removed after the training series, shRNA A/B mice spent a significantly shorter time in the correct quadrant (FIG. 3C, D). To further test neuronal network activity, recording electrodes were implanted into the entorhinal cortex to record electrocorticography (ECoG) signals from behaving mice. ECoG revealed in both experimental and control mice clear sleep and wakefulness patterns (FIG. 4A-B) and highlighted altered power spectrum patterns in the treated ones. When awake, shRNA A1+A2 mice showed significantly lower power in the delta (1-2.8 Hz) range, while during sleep, treated animals showed significantly higher power spectra at in the 9-12 Hz range within the EC (FIG. 4E). These data suggest markedly disturbed network activity during both awake and sleep.

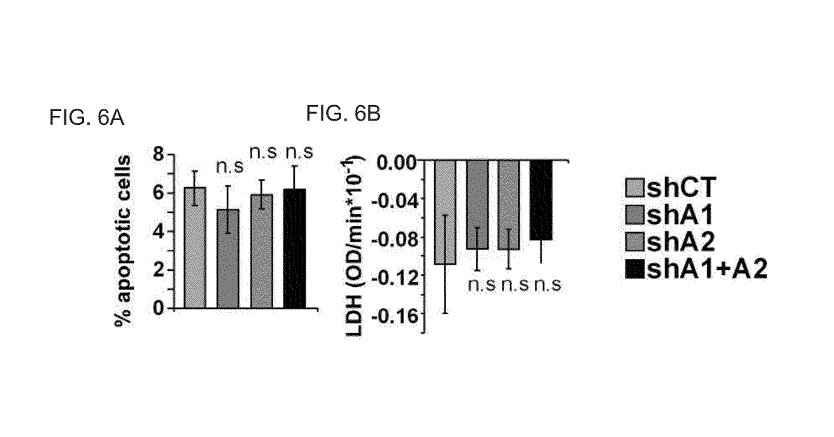

To investigate the cellular consequences of the hnRNP A/B decline, lentivirus-treated neuronal cultures were tested for synapse loss, the best correlate to the cognitive status in AD patients (Armstrong et al., 1991). As previously reported (Patry et al., 2003), hnRNP reduction did not induce overt cell death (FIG. 5A and FIG. 6). However, immuno-labeling of synapses and dendrites revealed that suppressing the to levels of hnRNP A1, A2/B1 or both in primary cultures dramatically reduced synaptic and dendritic density (FIG. 5B). Thus, synaptophysin-labelled synapses were reduced in total number (FIG. 5C), the remaining ones were smaller in size (FIG. 5E, F) and dendritic density (FIG. 5D) and acetylcholinesterase activity in the culture medium (FIG. 5G) were reduced in the knockdown neurons, mimicking several hallmarks of AD (Berson et al., 2008) Immuno-blots further demonstrated both pre- and post-synaptic deficits in the hnRNP A/B knockdown cultures (FIG. 5H). Moreover, hnRNP A/B reduction caused exon inclusion in several transcripts which were altered in the AD cortex (FIG. 5I). Other affected transcripts, including Reelin, SIPA1L1, and CD55 showed reduced exon inclusion, suggesting that the other factors may also contribute to the exon exclusion pattern observed in AD.

To establish whether β-amyloid and tau pathologies also affect hnRNP A/B expression in-vivo, the present inventors tested double transgenic APPsw/PS 1ΔE9 mice and mice expressing dually mutated K257T/P301S Tau for hnRNP A/B alterations. None of these strains presented such differences (FIG. 7A, B). Moreover, aging did not affect hnRNP A/B expression in either wild type or transgenic APPsw/PS1ΔE9 mice (FIG. 7A). Given that the AD brain is characterized by cholinergic deficits (Bartus et al., 1982), against which anti-cholinesterase therapies are directed (Querfurth and LaFerla, 2010), the present inventors further investigated whether cholinergic signaling can regulate neuronal hnRNP A/B levels. To induce in-vivo loss-of cholinergic function, cholinergic neurons were destroyed using intra-cerebroventricular (ICV) injections of a saporin-conjugated P75 antibody (muP75-sap) which selectively binds the P75 neurotrophin receptor on the surface of cholinergic neurons, inactivates their ribosomes when penetrating these cells, and causes learning and memory impairments in injected mice (Moreau et al., 2008). Entorhinal cortex samples harvested one month after saporin complex injections demonstrated reduced hnRNP A/B expression (FIG. 7C), showing a link between cholinergic cell death and hnRNP A/B loss. To test if this secondary loss in hnRNP A/B causes alternative splicing aberrations, the present inventors re-examined splicing events in CD55, SIPA1L1, Reelin and Dystonin. All of these transcripts exhibited increased exon inclusions in the cholinergic deficient mice, similarly to what was observed in the AD brain (FIG. 7D). Finally, a gain-of-function effect was induced by incubating primary neuronal cultures with 10 μM of the cholinergic agonist carbachol. Within 48 hours, this cholinergic excitation increased the expression of hnRNP A/B (FIG. 7E).

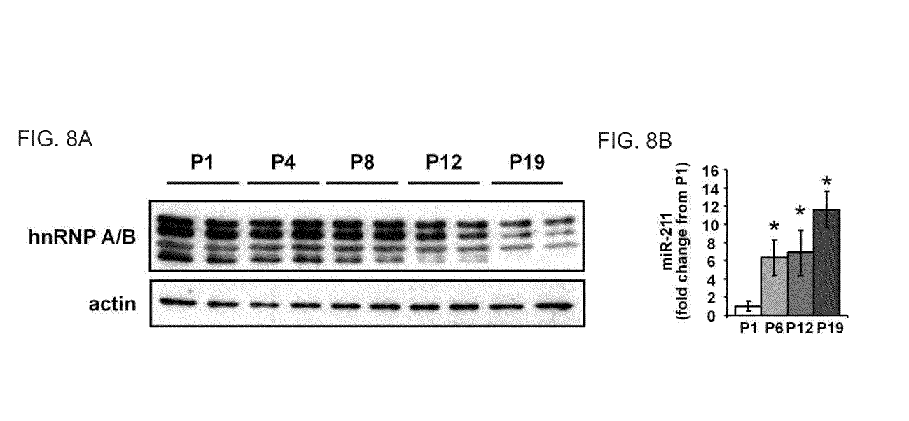

The present inventors next turned to study the molecular mechanism by which cholinergic transmission may regulate hnRNP A/B expression. Given the observed reduction in hnRNP A/B proteins, but not mRNA levels (FIG. 2) in the AD entorhinal cortex, and the high mRNA sequence similarities between the A/B family members, the present inventors tested whether the corresponding 3′ untranslated regions in the hnRNP A/B mRNAs contain “seed” regions to which common micro-RNAs (miRNA) may bind to repress their translation (FIG. 7F). Prediction algorithms (TargetScan, release 5.1) found several miRNAs that could potentially regulate all three hnRNPs A/B. Among those, the neuronal activity regulated miR-211 (Krol et al.), (FIG. 7G), but not the highly similar miR-204 was increased by 2-fold in the entorinal cortex of AD patients (FIG. 7H). Antisense oligonucleotides to miR-211, increased hnRNP A/B levels in cultured N9 cells (FIG. 7I), validating hnRNP A/B as mir-211 targets. Moreover, a negative correlation of hnRNPs A/B to miR-211 levels was found during mouse post-natal cortical development, further supporting the proposed regulatory role of miR-211 (FIGS. 8A-B), and suggesting that miRNA-211 increases in AD may directly contribute to hnRNP A/B loss. Intriguingly, miR-132, a well-established regulator of cholinergic gene expression (Shaked et al., 2009) and synaptic structure and function (Edbauer et al., 2010), was dramatically reduced in the AD entorhinal cortex (FIG. 7H). This may contribute to the AD-characteristic synaptic deficits (Armstrong et al., 1991), and may explain why AD cholinesterase levels are only marginally reduced (Berson et al., 2008) in spite of the massive loss of cholinergic neurons (Bartus et al., 1982). These results suggested that cholinergic signaling may regulate miRNA-211 and miRNA-132 expression. To critically challenge this hypothesis in an acute model, mice were injected with the muscarinic agonist. 48 hours after injections, increased levels of miRNA-132 and reduced levels of miRNA-211 were observed (FIG. 7J). Thus, reduced cholinergic signaling, as observed in AD, likely regulates hnRNPs A/B expression, at least in part by regulating miRNA-211. The inverse expression pattern of miRNA-132 and miRNA-211 suggested that at least one of them may negatively regulate the other. To test this possibility the present inventors recapitulated the expression pattern seen in AD in primary neurons by lenti-virus mediated overexpression of miRNA-211 and antisense olgonucleotide directed against miRNA-132. Measuring the levels of these miRNAs following 48 hours demonstrated that altered miRNA-211 levels had no effect on miRNA-132. However, knocking-down miRNA-132 elevated miRNA-211 by 3-folds (FIG. 7K). Thus, miRNA-132 likely mediates the effect of cholinergic signaling on miRNA-211 and therefore on hnRNP A/B expression.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

Armstrong, T. P., Hansen, L. A., Salmon, D. P., Masliah, E., Pay, M., Kunin, J. M., and Katzman, R. (1991). Rapidly progressive dementia in a patient with the Lewy body variant of Alzheimer's disease. Neurology 41, 1178-1180.
Ashe, K. H., and Zahs, K. R. (2010). Probing the biology of Alzheimer's disease in mice. Neuron 66, 631-645.
Bartus, R. T., Dean, R. L., 3rd, Beer, B., and Lippa, A. S. (1982). The cholinergic hypothesis of geriatric memory dysfunction. Science 217, 408-414.
Berson, A., Knobloch, M., Hanan, M., Diamant, S., Sharoni, M., Schuppli, D., Geyer, B. C., Ravid, R., Mor, T. S., Nitsch, R. M., and Soreq, H. (2008). Changes in readthrough acetylcholinesterase expression modulate amyloid-beta pathology. Brain 131, 109-119.
Chen, M., and Manley, J. L. (2009). Mechanisms of alternative splicing regulation: insights from molecular and genomics approaches. Nat Rev Mol Cell Biol 10, 741-754.
Cooper, T. A., Wan, L., and Dreyfuss, G. (2009). RNA and disease. Cell 136, 777-793.
Donev, R., Newall, A., Thome, J., and Sheer, D. (2007). A role for SC35 and hnRNPA1 in the determination of amyloid precursor protein isoforms. Mol Psychiatry 12, 681-690.
Du, H., Cline, M. S., Osborne, R. J., Tuttle, D. L., Clark, T. A., Donohue, J. P., Hall, M. P., Shiue, L., Swanson, M. S., Thornton, C. A., and Ares, M., Jr. (2010). Aberrant alternative splicing and extracellular matrix gene expression in mouse models of myotonic dystrophy. Nat Struct Mol Biol 17, 187-193.
Edbauer, D., Neilson, J. R., Foster, K. A., Wang, C. F., Seeburg, D. P., Batterton, M. N., Tada, T., Dolan, B. M., Sharp, P. A., and Sheng, M. (2010). Regulation of synaptic structure and function by FMRP-associated microRNAs miR-125b and miR-132. Neuron 65, 373-384.
Kabashi, E., Valdmanis, P. N., Dion, P., Spiegelman, D., McConkey, B. J., Vande Velde, C., Bouchard, J. P., Lacomblez, L., Pochigaeva, K., Salachas, F., et al. (2008). TARDBP mutations in individuals with sporadic and familial amyotrophic lateral sclerosis. Nat Genet 40, 572-574.
Kashima, T., Rao, N., David, C. J., and Manley, J. L. (2007). hnRNP A1 functions with specificity in repression of SMN2 exon 7 splicing. Hum Mol Genet 16, 3149-3159.
Kocherhans, S., Madhusudan, A., Doehner, J., Breu, K. S., Nitsch, R. M., Fritschy, J. M., and Knuesel, I. (2010). Reduced Reelin expression accelerates amyloid-beta plaque formation and tau pathology in transgenic Alzheimer's disease mice. J Neurosci 30, 9228-9240.
Krol, J., Busskamp, V., Markiewicz, I., Stadler, M. B., Ribi, S., Richter, J., Duebel, J., Bicker, S., Fehling, H. J., Schubeler, D., et al. (2010). Characterizing light-regulated retinal microRNAs reveals rapid turnover as a common property of neuronal microRNAs. Cell 141, 618-631.
Lefebvre, S., Burglen, L., Reboullet, S., Clermont, O., Burlet, P., Viollet, L., Benichou, B., Cruaud, C., Millasseau, P., Zeviani, M., and et al. (1995). Identification and characterization of a spinal muscular atrophy-determining gene. Cell 80, 155-165.
Licatalosi, D. D., and Darnell, R. B. (2006). Splicing regulation in neurologic disease. Neuron 52, 93-101.
Luo, L., and O'Leary, D. D. (2005). Axon retraction and degeneration in development and disease. Annu Rev Neurosci 28, 127-156.
Mayeda, A., and Krainer, A. R. (1992). Regulation of alternative pre-mRNA splicing by hnRNP Al and splicing factor SF2. Cell 68, 365-375.
Moreau, P. H., Cosquer, B., Jeltsch, H., Cassel, J. C., and Mathis, C. (2008). Neuroanatomical and behavioral effects of a novel version of the cholinergic immunotoxin mu p75-saporin in mice. Hippocampus 18, 610-622.
Pak, D. T., Yang, S., Rudolph-Correia, S., Kim, E., and Sheng, M. (2001). Regulation of dendritic spine morphology by SPAR, a PSD-95-associated RapGAP. Neuron 31, 289-303.
Park, S. M., Kim, K., Lee, E. J., Kim, B. K., Lee, T. J., Seo, T., Jang, I. S., Lee, S. H., Kim, S., Lee, J. H., and Park, J. (2009). Reduced expression of DRAM2/TMEM77 in tumor cells interferes with cell death. Biochem Biophys Res Commun 390, 1340-1344.
Pastorino, L., Sun, A., Lu, P. J., Zhou, X. Z., Balastik, M., Finn, G., Wulf, G., Lim, J., Li, S. H., Li, X., et al. (2006). The prolyl isomerase Pinl regulates amyloid precursor protein processing and amyloid-beta production. Nature 440, 528-534.
Patry, C., Bouchard, L., Labrecque, P., Gendron, D., Lemieux, B., Toutant, J., Lapointe, E., Wellinger, R., and Chabot, B. (2003). Small interfering RNA-mediated reduction in heterogeneous nuclear ribonucleoparticule Al/A2 proteins induces apoptosis in human cancer cells but not in normal mortal cell lines. Cancer Res 63, 7679-7688.
Pimplikar, S. W., Nixon, R. A., Robakis, N. K., Shen, J., and Tsai, L. H. (2010). Amyloid-independent mechanisms in Alzheimer's disease pathogenesis. J Neurosci 30, 14946-14954.
Querfurth, H. W., and LaFerla, F. M. (2010). Alzheimer's disease. N Engl J Med 362, 329-344.
Rosenmann, H., Grigoriadis, N., Eldar-Levy, H., Avital, A., Rozenstein, L., Touloumi, O., Behar, L., Ben-Hur, T., Avraham, Y., Berry, E., et al. (2008). A novel transgenic mouse expressing double mutant tau driven by its natural promoter exhibits tauopathy characteristics. Exp Neurol 212,71-84.
Shaked, I., Meerson, A., Wolf, Y., Avni, R., Greenberg, D., Gilboa-Geffen, A., and Soreq, H. (2009). MicroRNA-132 potentiates cholinergic anti-inflammatory signaling by targeting acetylcholinesterase. Immunity 31, 965-973.
Sofola, O. A., Jin, P., Qin, Y., Duan, R., Liu, H., de Haro, M., Nelson, D. L., and Botas, J. (2007). RNA-binding proteins hnRNP A2/B1 and CUGBP1 suppress fragile X CGG premutation repeat-induced neurodegeneration in a Drosophila model of FXTAS. Neuron 55, 565-571.
Sonnenberg, A., and Liem, R. K. (2007). Plakins in development and disease. Exp Cell Res 313, 2189-2203.
Sreedharan, J., Blair, I. P., Tripathi, V. B., Hu, X., Vance, C., Rogelj, B., Ackerley, S., Durnall, J. C., Williams, K. L., Buratti, E., et al. (2008). TDP-43 mutations in familial and sporadic amyotrophic lateral sclerosis. Science 319, 1668-1672.
Tai, H. C., and Schuman, E. M. (2008). Ubiquitin, the proteasome and protein degradation in neuronal function and dysfunction. Nat Rev Neurosci 9, 826-838.
Uehara, T., Nakamura, T., Yao, D., Shi, Z. Q., Gu, Z., Ma, Y., Masliah, E., Nomura, Y., and Lipton, S. A. (2006). S-nitrosylated protein-disulphide isomerase links protein misfolding to neurodegeneration. Nature 441, 513-517.
Vance, C., Rogelj, B., Hortobagyi, T., De Vos, K. J., Nishimura, A. L., Sreedharan, J., Hu, X., Smith, B., Ruddy, D., Wright, P., et al. (2009). Mutations in FUS, an RNA processing protein, cause familial amyotrophic lateral sclerosis type 6. Science 323, 1208-1211.
Villa, C., Fenoglio, C., De Riz, M., Clerici, F., Marcone, A., Benussi, L., Ghidoni, R., Gallone, S., Cortini, F., Serpente, M., et al. (2011). Role of hnRNP-A1 and miR-590-3p in Neuronal Death: Genetics and Expression Analysis in Patients with Alzheimer Disease and Frontotemporal Lobar Degeneration. Rejuvenation Res.
Wang, Y., Li, Y., Dalle Lucca, S. L., Simovic, M., Tsokos, G. C., and Dalle Lucca, J. J. (2010). Decay accelerating factor (CD55) protects neuronal cells from chemical hypoxia-induced injury. J Neuroinflammation 7, 24.
Xie, J., and Black, D. L. (2001). A CaMK IV responsive RNA element mediates depolarization-induced alternative splicing of ion channels. Nature 410, 936-939.
Zhang, C., Frias, M. A., Mele, A., Ruggiu, M., Eom, T., Marney, C. B., Wang, H., Licatalosi, D. D., Fak, J. J., and Darnell, R. B. (2010). Integrative modeling defines the Nova splicing-regulatory network and its combinatorial controls. Science 329, 439-443.
Zhang, Z., Lotti, F., Dittmar, K., Younis, I., Wan, L., Kasim, M., and Dreyfuss, G. (2008). SMN deficiency causes tissue-specific perturbations in the repertoire of snRNAs and widespread defects in splicing. Cell 133, 585-600.

1. (canceled)

2. A method of treating a neurodegenerative disease in a subject in need thereof comprising administering to the subject a therapeutically effective amount of an agent which upregulates an amount or activity of heterogeneous nuclear ribonucleoproteins A/B (hnRNP A/B) in a brain of the subject, thereby treating the neurodegenerative disease.

3. The method of claim 2, wherein said hnRNP A/B polypeptide is hnRNP Al and/or A2/B1.

4. The method of claim 2, wherein said agent comprises a polynucleotide agent.

5. The method of claim 4, wherein said polynucleotide agent comprises a miRNA.

6. The method of claim 5, wherein said miRNA is encoded by a sequence as set forth in SEQ ID NO: 86.

7. The method of claim 4, wherein said polynucleotide agent comprises a miRNA antagonist.

8. The method of claim 7, wherein said miRNA antagonist comprises a miR211 antagonist.

9. The method of claim 8, wherein said miR211 antagonist comprises a sequence as set forth in SEQ ID NO: 87.

10. The method of claim 2, wherein said neurodegenerative disease is selected from the group consisting of Alzheimer's disease, epilepsy, amyotrophic lateral sclerosis, stroke, autoimmune encephalomyelitis, diabetic neuropathy and glaucomatous neuropathy.

11. The method of claim 2, wherein said neurodegenerative disease is Alzheimer's disease.

12. A pharmaceutical composition comprising miR132 or a miR211 antagonist or both as an active agent and a pharmaceutically acceptable carrier.

13. The pharmaceutical composition of claim 12, being formulated for crossing the blood brain barrier.

14. The pharmaceutical composition of claim 12, wherein said miR211 antagonist comprises a sequence as set forth in SEQID NO: 87.


SEQUENCE LISTING (93 sequences)

SEQ ID NO: 1 - DNA, 19 bp, Artificial sequence
  Single strand DNA oligonucleotide
ttctcccttctccctctgc

SEQ ID NO: 2 - DNA, 18 bp, Artificial sequence
  Single strand DNA oligonucleotide
gtggtcccaagacaatgc

SEQ ID NO: 3 - DNA, 20 bp, Artificial sequence
  Single strand DNA oligonucleotide
cctgcctctgttgagactcc

SEQ ID NO: 4 - DNA, 27 bp, Artificial sequence
  Single strand DNA oligonucleotide
ttctgctaacagtattctttaatgtga

SEQ ID NO: 5 - DNA, 21 bp, Artificial sequence
  Single strand DNA oligonucleotide
tgaagagtttgattccgaacg

SEQ ID NO: 6 - DNA, 18 bp, Artificial sequence
  Single strand DNA oligonucleotide
agccagggtatggctgct

SEQ ID NO: 7 - DNA, 20 bp, Artificial sequence
  Single strand DNA oligonucleotide
cagcctgactggacacagaa

SEQ ID NO: 8 - DNA, 20 bp, Artificial sequence
  Single strand DNA oligonucleotide
atctactgtgcccagcgact

SEQ ID NO: 9 - DNA, 24 bp, Artificial sequence
  Single strand DNA oligonucleotide
gaaaggatcatctctactttctgg

SEQ ID NO: 10 - DNA, 19 bp, Artificial sequence
  Single strand DNA oligonucleotide
gtggcgagatgctagacag

SEQ ID NO: 11 - DNA, 24 bp, Artificial sequence
  Single strand DNA oligonucleotide
attttgaacttgagcaggtagttg

SEQ ID NO: 12 - DNA, 20 bp, Artificial sequence
  Single strand DNA oligonucleotide
gggagagagagcgcagaata

SEQ ID NO: 13 - DNA, 18 bp, Artificial sequence
  Single strand DNA oligonucleotide
aagcctggctcagaagca

SEQ ID NO: 14 - DNA, 20 bp, Artificial sequence
  Single strand DNA oligonucleotide
tcatgagactgggatgtggt

SEQ ID NO: 15 - DNA, 21 bp, Artificial sequence
  Single strand DNA oligonucleotide
tgaagaagactccacgagagg

SEQ ID NO: 16 - DNA, 20 bp, Artificial sequence
  Single strand DNA oligonucleotide
gtgggaaaccatctcgaaag

SEQ ID NO: 17 - DNA, 21 bp, Artificial sequence
  Single strand DNA oligonucleotide
aaatccaggtctcgtgtcatc

SEQ ID NO: 18 - DNA, 23 bp, Artificial sequence
  Single strand DNA oligonucleotide
gagactggaattttcaagggact

SEQ ID NO: 19 - DNA, 20 bp, Artificial sequence
  Single strand DNA oligonucleotide
ctcggtggagcataaagagc

SEQ ID NO: 20 - DNA, 21 bp, Artificial sequence
  Single strand DNA oligonucleotide
tctccatcagtggaggaatca

SEQ ID NO: 21 - DNA, 20 bp, Artificial sequence
  Single strand DNA oligonucleotide
gcagtgtatggcaagggaac

SEQ ID NO: 22 - DNA, 20 bp, Artificial sequence
  Single strand DNA oligonucleotide
actacactgtgggggctgat

SEQ ID NO: 23 - DNA, 19 bp, Artificial sequence
  Single strand DNA oligonucleotide
ggatttgtgatgctggacg

SEQ ID NO: 24 - DNA, 20 bp, Artificial sequence
  Single strand DNA oligonucleotide
taggtttgaggggaagctca

SEQ ID NO: 25 - DNA, 22 bp, Artificial sequence
  Single strand DNA oligonucleotide
gaagtagagagatttgccatcg

SEQ ID NO: 26 - DNA, 23 bp, Artificial sequence
  Single strand DNA oligonucleotide
atcatgacatctgtgcttttcaa

SEQ ID NO: 27 - DNA, 20 bp, Artificial sequence
  Single strand DNA oligonucleotide
atggctgaacatttccaagg

SEQ ID NO: 28 - DNA, 20 bp, Artificial sequence
  Single strand DNA oligonucleotide
tgtgaccctggattttctgg

SEQ ID NO: 29 - DNA, 20 bp, Artificial sequence
  Single strand DNA oligonucleotide
aaaaccagggccttaccact

SEQ ID NO: 30 - DNA, 22 bp, Artificial sequence
  Single strand DNA oligonucleotide
gcttggaattttctaccaacca

SEQ ID NO: 31 - DNA, 22 bp, Artificial sequence
  Single strand DNA oligonucleotide
agaggagtagacagtgctgtgg

SEQ ID NO: 32 - DNA, 20 bp, Artificial sequence
  Single strand DNA oligonucleotide
tcagtcagatgggaactcca

SEQ ID NO: 33 - DNA, 22 bp, Artificial sequence
  Single strand DNA oligonucleotide
ttcttctgaccattgctcttga

SEQ ID NO: 34 - DNA, 21 bp, Artificial sequence
  Single strand DNA oligonucleotide
ctccaacagatgtcactgtgc

SEQ ID NO: 35 - DNA, 22 bp, Artificial sequence
  Single strand DNA oligonucleotide
ggatttaattgcagttggtggt

SEQ ID NO: 36 - DNA, 20 bp, Artificial sequence
  Single strand DNA oligonucleotide
ggatcatgtctgaccaccag

SEQ ID NO: 37 - DNA, 20 bp, Artificial sequence
  Single strand DNA oligonucleotide
aggtggagcaatccagatga

SEQ ID NO: 38 - DNA, 20 bp, Artificial sequence
  Single strand DNA oligonucleotide
gaggaatttgtgaccctgga

SEQ ID NO: 39 - DNA, 20 bp, Artificial sequence
  Single strand DNA oligonucleotide
ttgatggttcttttgcctga

SEQ ID NO: 40 - DNA, 20 bp, Artificial sequence
  Single strand DNA oligonucleotide
aaaccagaacaagggtgtgg

SEQ ID NO: 41 - DNA, 20 bp, Artificial sequence
  Single strand DNA oligonucleotide
ctctgctgtcaggcttgttg

SEQ ID NO: 42 - DNA, 20 bp, Artificial sequence
  Single strand DNA oligonucleotide
cggagaaaatcagcggtcta

SEQ ID NO: 43 - DNA, 21 bp, Artificial sequence
  Single strand DNA oligonucleotide
cgtgtactcaacaggaacgtg

SEQ ID NO: 44 - DNA, 21 bp, Artificial sequence
  Single strand DNA oligonucleotide
actgcagtaacactccaccat

SEQ ID NO: 45 - DNA, 22 bp, Artificial sequence
  Single strand DNA oligonucleotide
aaactgccgcaatatttagcat

SEQ ID NO: 46 - DNA, 23 bp, Artificial sequence
  Single strand DNA oligonucleotide
tccaactacagaagtctcaccaa

SEQ ID NO: 47 - DNA, 20 bp, Artificial sequence
  Single strand DNA oligonucleotide
tggttgtcctggaaacaggt

SEQ ID NO: 48 - DNA, 20 bp, Artificial sequence
  Single strand DNA oligonucleotide
gcagctctgtccagtggagt

SEQ ID NO: 49 - DNA, 19 bp, Artificial sequence
  Single strand DNA oligonucleotide
ggtcacgttccccttgaat

SEQ ID NO: 50 - DNA, 20 bp, Artificial sequence
  Single strand DNA oligonucleotide
ggcagaacacccagtcagat

SEQ ID NO: 51 - DNA, 19 bp, Artificial sequence
  Single strand DNA oligonucleotide
atgctgctcgaggacaaaa

SEQ ID NO: 52 - DNA, 20 bp, Artificial sequence
  Single strand DNA oligonucleotide
ttttggggctgatgagaatc

SEQ ID NO: 53 - DNA, 20 bp, Artificial sequence
  Single strand DNA oligonucleotide
ggaccgaacctctcagtgtc

SEQ ID NO: 54 - DNA, 20 bp, Artificial sequence
  Single strand DNA oligonucleotide
cccagcatgtacctgttacc

SEQ ID NO: 55 - DNA, 21 bp, Artificial sequence
  Single strand DNA oligonucleotide
tcacatgcaaaactgtcaagg

SEQ ID NO: 56 - DNA, 20 bp, Artificial sequence
  Single strand DNA oligonucleotide
gccacagcaaaaccttcatt

SEQ ID NO: 57 - DNA, 20 bp, Artificial sequence
  Single strand DNA oligonucleotide
aacaggtacatgctgggttg

SEQ ID NO: 58 - DNA, 20 bp, Artificial sequence
  Single strand DNA oligonucleotide
tgtctctgttgctgctgtcc

SEQ ID NO: 59 - DNA, 20 bp, Artificial sequence
  Single strand DNA oligonucleotide
tgctcagcaaacttggagtg

SEQ ID NO: 60 - DNA, 22 bp, Artificial sequence
  Single strand DNA oligonucleotide
tgattcaaggttcacactcaca

SEQ ID NO: 61 - DNA, 20 bp, Artificial sequence
  Single strand DNA oligonucleotide
aaaactgaggccttgctgaa

SEQ ID NO: 62 - DNA, 20 bp, Artificial sequence
  Single strand DNA oligonucleotide
ttcagcaaggcctcagtttt

SEQ ID NO: 63 - DNA, 21 bp, Artificial sequence
  Single strand DNA oligonucleotide
tcaggaggtattgtccctgtg

SEQ ID NO: 64 - DNA, 20 bp, Artificial sequence
  Single strand DNA oligonucleotide
cggcagtaagtcacctgtca

SEQ ID NO: 65 - DNA, 20 bp, Artificial sequence
  Single strand DNA oligonucleotide
cttcagctttgccagctctt

SEQ ID NO: 66 - DNA, 22 bp, Artificial sequence
  Single strand DNA oligonucleotide
gattcaagaccatcaggttgtg

SEQ ID NO: 67 - DNA, 21 bp, Artificial sequence
  Single strand DNA oligonucleotide
caatgttggccctaaatagaa

SEQ ID NO: 68 - DNA, 20 bp, Artificial sequence
  Single strand DNA oligonucleotide
tcaggcatgcagttcttttg

SEQ ID NO: 69 - DNA, 20 bp, Artificial sequence
  Single strand DNA oligonucleotide
gaaagcaggcagtaccttcg

SEQ ID NO: 70 - DNA, 20 bp, Artificial sequence
  Single strand DNA oligonucleotide
tagtgtggacgctgctgtct

SEQ ID NO: 71 - DNA, 20 bp, Artificial sequence
  Single strand DNA oligonucleotide
ggctctgtggtcaccagaat

SEQ ID NO: 72 - DNA, 19 bp, Artificial sequence
  Single strand DNA oligonucleotide
atggcatttcccccattag

SEQ ID NO: 73 - DNA, 20 bp, Artificial sequence
  Single strand DNA oligonucleotide
ggaggttggttttgcttcaa

SEQ ID NO: 74 - DNA, 20 bp, Artificial sequence
  Single strand DNA oligonucleotide
gagcgggacaaagttcaaaa

SEQ ID NO: 75 - DNA, 18 bp, Artificial sequence
  Single strand DNA oligonucleotide
cccgtccctcagatcctc

SEQ ID NO: 76 - DNA, 20 bp, Artificial sequence
  Single strand DNA oligonucleotide
atcatgtccgaccaccagtt

SEQ ID NO: 77 - DNA, 20 bp, Artificial sequence
  Single strand DNA oligonucleotide
ggcaatccagacaaagctga

SEQ ID NO: 78 - DNA, 20 bp, Artificial sequence
  Single strand DNA oligonucleotide
gcagtgccagactttcctct

SEQ ID NO: 79 - DNA, 20 bp, Artificial sequence
  Single strand DNA oligonucleotide
gcctcccatctttgttgaaa

SEQ ID NO: 80 - DNA, 19 bp, Artificial sequence
  Single strand DNA oligonucleotide
ggcaaccccacctactacg

SEQ ID NO: 81 - DNA, 20 bp, Artificial sequence
  Single strand DNA oligonucleotide
gactggatgcttgtcgaggt

SEQ ID NO: 82 - DNA, 20 bp, Artificial sequence
  Single strand DNA oligonucleotide
aggtcccaccaacagttcag

SEQ ID NO: 83 - DNA, 20 bp, Artificial sequence
  Single strand DNA oligonucleotide
cgagactgcagtgagctacg

SEQ ID NO: 84 - DNA, 20 bp, Artificial sequence
  Single strand DNA oligonucleotide
gcgctagtcggtctggtaag

SEQ ID NO: 85 - DNA, 20 bp, Artificial sequence
  Single strand DNA oligonucleotide
aaactgaggccttgctgaaa

SEQ ID NO: 86 - RNA, 22 nt, Homo sapiens
  misc_feature: miR132
uaacagucuacagccauggucg

SEQ ID NO: 87 - DNA, 22 bp, Artificial sequence
  miR211 antagonist
aggcgaaggatgacaaagggaa

SEQ ID NO: 88 - RNA, 22 nt, Homo sapiens
  misc_feature: hnRNPA1 common seed region complementary to miR-211
ugaaguucaccauaaaagggau

SEQ ID NO: 89 - RNA, 23 nt, Homo sapiens
  misc_feature: hnRNPA2 common seed region complementary to miR-211
gcacucuuuaaaauaaaagggaa

SEQ ID NO: 90 - RNA, 23 nt, Homo sapiens
  misc_feature: hnRNPA3 common seed region complementary to miR-211
ccauuuaaauucugaaaagggau

SEQ ID NO: 91 - RNA, 22 nt, Homo sapiens
  misc_feature: miRNA 211 sequence
uccgcuuccuacuguuucccuu

SEQ ID NO: 92 - DNA, 3666 bp, Homo sapiens
agtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggagagagaaaaggaacagttccgtaagctctttattggtggcttaagctttgaaaccacagaagaaagtttgaggaactactacgaacaatggggaaagcttacagactgtgtggtaatgagggatcctgcaagcaaaagatcaagaggatttggttttgtaactttttcatccatggctgaggttgatgctgccatggctgcaagacctcattcaattgatgggagagtagttgagccaaaacgtgctgtagcaagagaggaatctggaaaaccaggggctcatgtaactgtgaagaagctgtttgttggcggaattaaagaagatactgaggaacatcaccttagagattactttgaggaatatggaaaaattgataccattgagataattactgataggcagtctggaaagaaaagaggctttggctttgttacttttgatgaccatgatcctgtggataaaatcgtattgcagaaataccataccatcaatggtcataatgcagaagtaagaaaggctttgtctagacaagaaatgcaggaagttcagagttctaggagtggaagaggaggcaactttggctttggggattcacgtggtggcggtggaaatttcggaccaggaccaggaagtaactttagaggaggatctgatggatatggcagtggacgtggatttggggatggctataatgggtatggaggaggacctggaggtggcaattttggaggtagccccggttatggaggaggaagaggaggatatggtggtggaggacctggatatggcaaccagggtgggggctacggaggtggttatgacaactatggaggaggaaattatggaagtggaaattacaatgattttggaaattataaccagcaaccttctaactacggtccaatgaagagtggaaactttggtggtagcaggaacatggggggaccatatggtggaggaaactatggtccaggaggcagtggaggaagtgggggttatggtgggaggagccgatactgagcttcttcctatttgccatgggcttcactgtataaataggagaggatgagagcccagaggtaacagaacagcttcaggttatcgaaataacaatgttaaggaaactcttatctcagtcatgcataaatatgcagtgatatggcagaagacaccagagcagatgcagagagccattttgtgaatggattggattatttaataacattaccttactgtggaggaaggattgtaaaaaaaaatgcctttgagacagtttcttagctttttaattgttgtttctttctagtggtctttgtaagagtgtagaagcattccttctttgataatgttaaatttgtaagtttcaggtgacatgtgaaaccttttttaagatttttctcaaagttttgaaaagctattagccaggatcatggtgtaataagacataacgtttttcctttaaaaaaatttaagtgcgtgtgtagagttaagaagctgttgtacatttatgatttaataaaataattctaaaggaaattgtgtaattatagactttttattttaaataagttaaggagtgggtagtataattaaggtccgttgcaaagctgttgttatatttgtataagataaatgctggtcagatgtaagtgtgttgtctgcaattcatcaggattaaattatgtagataacttaagggatatctctgcaaggagaaacacctttttagatcttttagatgctgcttcttcaatgcaaggaaaggaaataaccccagcgaggtactcttcagggacacaggtctagtacaagagaactcttgacggctactaagttcagccagtcttaaaaaactgtgctgtttctacaaaactttaactacagtagtttataaggatgccaacgaaagctgagggtgtagagcaaaatagttctaagcttcagttaaacttctttaggtaagatcttatttacttttcctttcttaattttcctccctaaaagataaactaatactcttaaatggtctttcagtatagtggttcttacgtagtttaacatagctataaattgagtttaacaatttataaactcaagagaataatttttataaaccctgttttccaatctgtcatttacttaaattattttggttgtttttccctttttttccttcttttcccaccccctccccctccatgtgaagatttgggtgcttaacatatcatttttttccctgccggaattttagcattgatatgaaccatggacaagtatattctgctgccacaaagactgtaaagtgcttcatttcaacagctgaggcaagccaagtgatcattaataaagcttttcttggttccttcagtggtgttggtagtaaaatggtaggtaaaagttaggctgcaagttcaataaatcatgagatttcccatcgttacacccttgtgtattcacatttcttggatcaaacattttgagtgaactaggggtttttattaaagacatttgttgtatttatggttgtaactgtacatgcttatcaggatgagactgaaagaaggtagggcaaaaatggttgaatctattttcagatagtagttcatacttgagtgaagtgtcttgtctgcattatgaagcctggtatgtatccagtactaaataggtgggttaaatgtggtaattctagttcagtgtcttaccctgaagagaaagttgtaggttggctgttgaaattcattccttagatatgatcagtttgattgcccggctttattgcctttacaggaatgtgatactcagggcttactctatacaccaatgagtcttctttgatcctaagaccaccactgaagttgtttaggttcttttggacaaacatgataaacttcttcagatactttttttttcctttggcaggaaggtgtcttgctgcaggtaactaatgaagaagtggtcaaccacagagtcttcaagaaataagaaattctgtaccatctgaaagtagttcttgttggtgccttcatttaaaaagcactctttaaaataaaagggaaatgttttctgataaaacaaacatttagttgaggttcttgatataaaacaattacaaaatgagtgttgtttgtaaaacagtaacatcaaattggctagagagataaatgtatcatgttttaaattaggttttgtgagtagacagattacaattctattttaaatataaagtttataaaataaatactttttgtatccaaatacttggtgtaatgtttacacataaaatgtgtgaatcttgttctataaatatttggttgtctaaaagatcaccatcccctaaatttttaaaagcagtttcacaaagctatgcatattttaatattaacaggtaaatgagaagagcattgtggacattattggctgtccccaataaaatgctgttcatta

SEQ ID NO: 93 - DNA, 3702 bp, Homo sapiens
agtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggagaaaactttagaaactgttcctttggagaggaaaaagagagaaaaggaacagttccgtaagctctttattggtggcttaagctttgaaaccacagaagaaagtttgaggaactactacgaacaatggggaaagcttacagactgtgtggtaatgagggatcctgcaagcaaaagatcaagaggatttggttttgtaactttttcatccatggctgaggttgatgctgccatggctgcaagacctcattcaattgatgggagagtagttgagccaaaacgtgctgtagcaagagaggaatctggaaaaccaggggctcatgtaactgtgaagaagctgtttgttggcggaattaaagaagatactgaggaacatcaccttagagattactttgaggaatatggaaaaattgataccattgagataattactgataggcagtctggaaagaaaagaggctttggctttgttacttttgatgaccatgatcctgtggataaaatcgtattgcagaaataccataccatcaatggtcataatgcagaagtaagaaaggctttgtctagacaagaaatgcaggaagttcagagttctaggagtggaagaggaggcaactttggctttggggattcacgtggtggcggtggaaatttcggaccaggaccaggaagtaactttagaggaggatctgatggatatggcagtggacgtggatttggggatggctataatgggtatggaggaggacctggaggtggcaattttggaggtagccccggttatggaggaggaagaggaggatatggtggtggaggacctggatatggcaaccagggtgggggctacggaggtggttatgacaactatggaggaggaaattatggaagtggaaattacaatgattttggaaattataaccagcaaccttctaactacggtccaatgaagagtggaaactttggtggtagcaggaacatggggggaccatatggtggaggaaactatggtccaggaggcagtggaggaagtgggggttatggtgggaggagccgatactgagcttcttcctatttgccatgggcttcactgtataaataggagaggatgagagcccagaggtaacagaacagcttcaggttatcgaaataacaatgttaaggaaactcttatctcagtcatgcataaatatgcagtgatatggcagaagacaccagagcagatgcagagagccattttgtgaatggattggattatttaataacattaccttactgtggaggaaggattgtaaaaaaaaatgcctttgagacagtttcttagctttttaattgttgtttctttctagtggtctttgtaagagtgtagaagcattccttctttgataatgttaaatttgtaagtttcaggtgacatgtgaaaccttttttaagatttttctcaaagttttgaaaagctattagccaggatcatggtgtaataagacataacgtttttcctttaaaaaaatttaagtgcgtgtgtagagttaagaagctgttgtacatttatgatttaataaaataattctaaaggaaattgtgtaattatagactttttattttaaataagttaaggagtgggtagtataattaaggtccgttgcaaagctgttgttatatttgtataagataaatgctggtcagatgtaagtgtgttgtctgcaattcatcaggattaaattatgtagataacttaagggatatctctgcaaggagaaacacctttttagatcttttagatgctgcttcttcaatgcaaggaaaggaaataaccccagcgaggtactcttcagggacacaggtctagtacaagagaactcttgacggctactaagttcagccagtcttaaaaaactgtgctgtttctacaaaactttaactacagtagtttataaggatgccaacgaaagctgagggtgtagagcaaaatagttctaagcttcagttaaacttctttaggtaagatcttatttacttttcctttcttaattttcctccctaaaagataaactaatactcttaaatggtctttcagtatagtggttcttacgtagtttaacatagctataaattgagtttaacaatttataaactcaagagaataatttttataaaccctgttttccaatctgtcatttacttaaattattttggttgtttttccctttttttccttcttttcccaccccctccccctccatgtgaagatttgggtgcttaacatatcatttttttccctgccggaattttagcattgatatgaaccatggacaagtatattctgctgccacaaagactgtaaagtgcttcatttcaacagctgaggcaagccaagtgatcattaataaagcttttcttggttccttcagtggtgttggtagtaaaatggtaggtaaaagttaggctgcaagttcaataaatcatgagatttcccatcgttacacccttgtgtattcacatttcttggatcaaacattttgagtgaactaggggtttttattaaagacatttgttgtatttatggttgtaactgtacatgcttatcaggatgagactgaaagaaggtagggcaaaaatggttgaatctattttcagatagtagttcatacttgagtgaagtgtcttgtctgcattatgaagcctggtatgtatccagtactaaataggtgggttaaatgtggtaattctagttcagtgtcttaccctgaagagaaagttgtaggttggctgttgaaattcattccttagatatgatcagtttgattgcccggctttattgcctttacaggaatgtgatactcagggcttactctatacaccaatgagtcttctttgatcctaagaccaccactgaagttgtttaggttcttttggacaaacatgataaacttcttcagatactttttttttcctttggcaggaaggtgtcttgctgcaggtaactaatgaagaagtggtcaaccacagagtcttcaagaaataagaaattctgtaccatctgaaagtagttcttgttggtgccttcatttaaaaagcactctttaaaataaaagggaaatgttttctgataaaacaaacatttagttgaggttcttgatataaaacaattacaaaatgagtgttgtttgtaaaacagtaacatcaaattggctagagagataaatgtatcatgttttaaattaggttttgtgagtagacagattacaattctattttaaatataaagtttataaaataaatactttttgtatccaaatacttggtgtaatgtttacacataaaatgtgtgaatcttgttctataaatatttggttgtctaaaagatcaccatcccctaaatttttaaaagcagtttcacaaagctatgcatattttaatattaacaggtaaatgagaagagcattgtggacattattggctgtccccaataaaatgctgttcatta